(19) United States
(12) Patent Application Publication
(10) Pub. No.: US 2020/0299242 A1
(43) Pub. Date: Sep. 24, 2020

(54) PROCESS FOR THE PREPARATION OF ROXADUSTAT AND ITS INTERMEDIATES

(71) Applicant: DR. REDDY'S LABORATORIES LIMITED, Hyderabad (IN)

(72) Inventors: Amarnath Reddy LEKKALA, Hyderabad (IN); Jaydeepkumar DAHYABHAI LILAKAR, Lilapor (IN); Mohammad AASEEF, Gudivada (IN); Rehani Rajeev BUDHDEV, Hyderabad (IN); Sekhar Munaswamy NARIYAM, Hyderabad (IN); Rakeshwar BANDICHHOR, Chandipur (IN); Sharad Santu PACHORE, Bahadarabad (IN); Soma Rani SARKAR, Hyderabad (IN); Babu IRENI, Hyderabad (IN); Deep MALA, Raja Ka Tajpur (IN); Kiran Kumar DONIPARTHI, Nellore (IN); Venkata Krishna Rao BADARLA, Hyderabad (IN)

(21) Appl. No.: 16/768,523

(22) PCT Filed: Nov. 30, 2018
(86) PCT No.: PCT/IB2018/059504

§ 371 Date: May 29, 2020

(30) Foreign Application Priority Data
Dec. 1, 2017 (IN) 201741043126
May 25, 2018 (IN) 201841019627

(51) Int. Cl. C07D 217/24; C07D 233/64; C07D 263/34; C07C 65/21

(52) U.S. Cl. CPC C07D 217/24; C07C 65/21; C07D 263/34; C07D 233/64

(57) ABSTRACT

The present invention provides the process for the preparation of Roxadustat and its intermediates. Another aspect of the present invention provides a process for preparation of ethyl-5-(2-butoxycarbonyl)-4-phenoxyphenyl) oxazole-4-carboxylate of the formula (X) and its use in the preparation of Roxadustat. Another aspect of the present invention provides a process for the preparation of ethyl-4-hydroxy-1-methyl-7-phenoxyisoquinoline-3-carboxylate of the formula (XIII) and its use in the preparation of Roxadustat.

### FIELD OF THE APPLICATION

The present invention provides the process for the preparation of Roxadustat and its intermediates. Another aspect of the present invention provides a process for preparation of ethyl-5-(2-butoxycarbonyl)-4-phenoxyphenyl) oxazole-4-carboxylate of the formula (X) and its use in the preparation of Roxadustat. Another aspect of the present invention provides a process for the preparation of ethyl-4-hydroxy-1 -methyl-7-phenoxyisoquinoline-3-carboxylate of the formula (XIII) and its use in the preparation of Roxadustat.

### BACKGROUND

Roxadustat (I) or FG-4592 is chemically known as [(4-Hydroxy-1-methyl-7-phenoxy-iso quinoline-3-carbonyl)-amino]-acetic acid. It is an oral small molecule inhibitor of HIF prolyl hydroxylases, or HIF-PHs, in Phase 3 clinical development for treating and preventing disorders associated with HIF, including anemia in chronic kidney disease, or CKD, ischemia, and hypoxia.

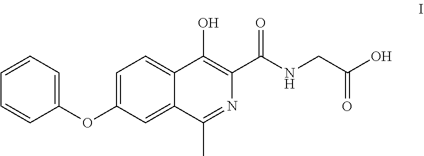 [SMILES: Cc1nc(C(=O)NCC(=O)O)c(O)c2ccc(Oc3ccccc3)cc12]

Product patent of Roxadustat (U.S. Pat. No. 7,323,475 B2) discloses a process for the preparation of Roxadustat in Example D-81.

Several processes for the preparation of Roxadustat and its intermediates have been disclosed in WO2014014834A1, U.S. Pat. No. 9,206,134B2 and WO2018072662A1.

In view of the importance of treating and preventing disorders associated with HIF, including anemia in chronic kidney disease, cost-effective and novel methods of making such drugs and their intermediates are always of interest. The present invention provides a cost and yield-improving process to prepare Roxadustat (I) and its intermediates thereof.

### SUMMARY OF THE INVENTION

The present application provides a synthetic processes for obtaining Roxadustat of formula (I) and its related intermediates.

In a first embodiment of the present invention provides a process for the preparation of Roxadustat (I) or its pharmaceutically acceptable salts, which comprises;
a) converting a compound of formula (II) to compound of formula (III);

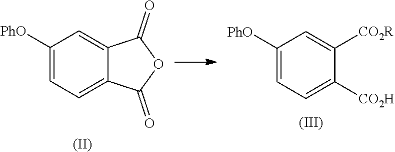

wherein R is C₁-C₆ alkyl;
b) optionally purifying a compound of formula (III);
c) treating a compound of formula (III) with alkyl 2-isocyanoacetate (IV) to form a compound of formula (V);

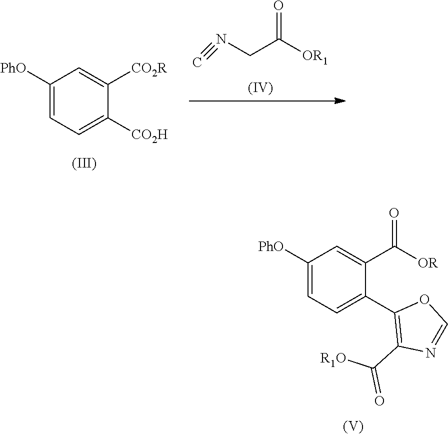

wherein R is C₁-C₆ alkyl and R₁ is H, C₂-C₆ alkyl;
d) converting a compound of formula (V) to form a compound of formula (VI);

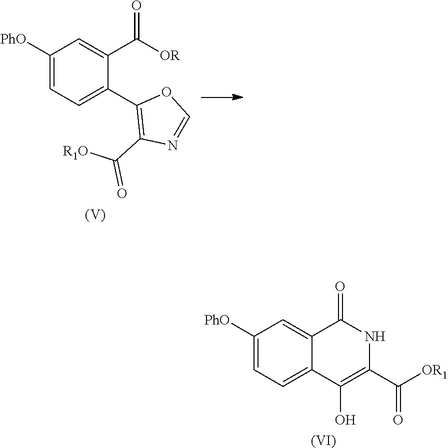

wherein R is C₁-C₆ alkyl and R₁ is H, C₂-C₆ alkyl;
e) halogenation of a compound of formula (VI) to form a compound of formula (VII);

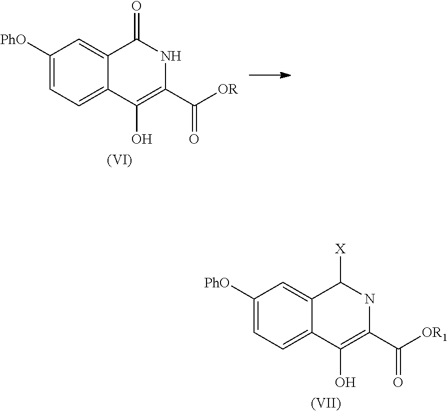

wherein R₁ is H, C₂-C₆ alkyl; X is Cl, Br, I;
f) converting a compound of formula (VII) to a compound of formula (VIII);

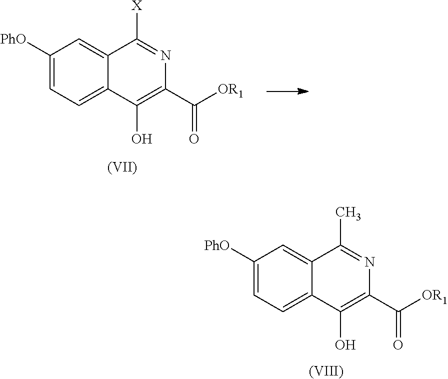

wherein R₁ is H, C₂-C₆ alkyl; X is Cl, Br, I;
g) treating a compound of formula (VIII) with glycine to form Roxadustat (I) or its pharmaceutically acceptable salts.

In a second embodiment of the present invention provides a process for the preparation of Roxadustat (I) or its pharmaceutically acceptable salts, which comprises;
a) converting a compound of formula (II) to compound of formula (IX);

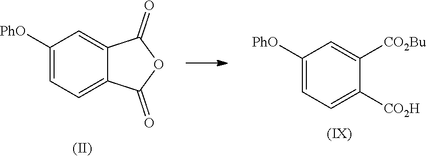

b) optionally purifying a compound of formula (IX);
c) treating a compound of formula (IX) with ethyl 2-isocyanoaceate to form a compound of formula (X);

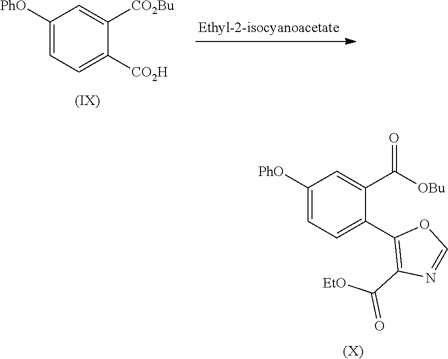

d) converting a compound of formula (X) to form a compound of formula (XI);

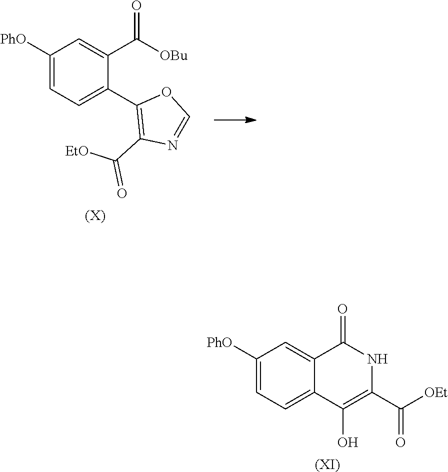

e) halogenation of a compound of formula (XI) to form a compound of formula (XII);

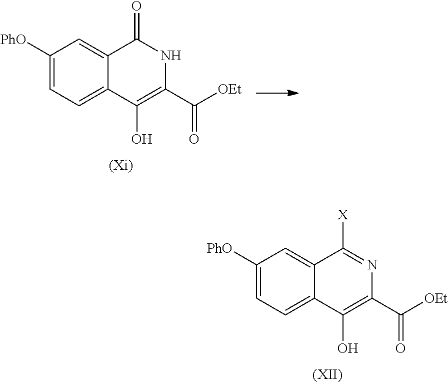

f) converting a compound of formula (XII) to a compound of formula (XIII);

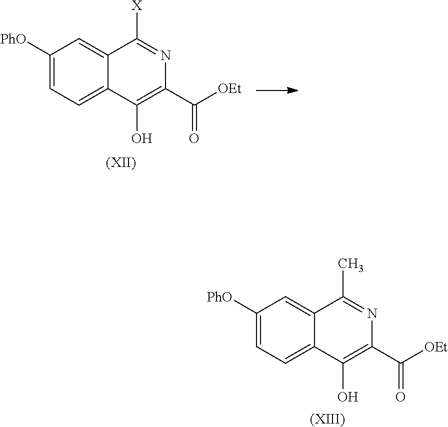

g) treating a compound of formula (XIII) with glycine to form Roxadustat (I) or its pharmaceutically acceptable salts.

In a third embodiment of the present invention provides a process for the preparation of Roxadustat (I) or its pharmaceutically acceptable salts, which comprises;
a) converting a compound of formula (III) to a compound of formula (IIIa);

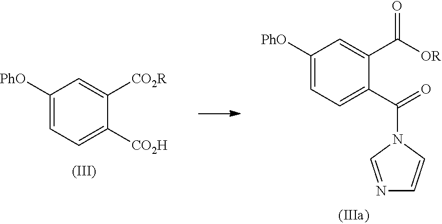

wherein R is C₁-C₆ alkyl;
b) treating a compound of formula (IIIa) with alkyl 2-isocyanoacetate (IV) to form a compound of formula (V);

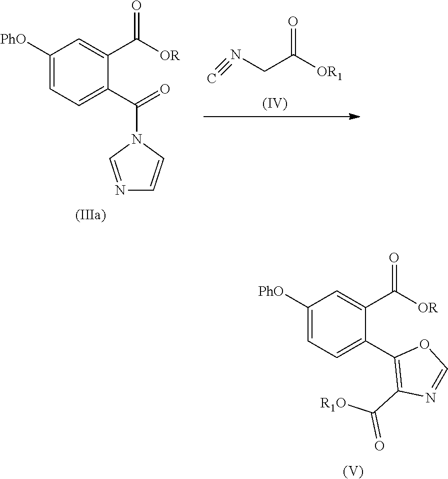

wherein R is C₁-C₆ alkyl and R₁ is H, C₂-C₆ alkyl;
c) converting a compound of formula (V) to Roxadustat (I) or its pharmaceutically acceptable salts.

In a fourth embodiment of the present invention provides a process for the preparation of Roxadustat (I) or its pharmaceutically acceptable salts, which comprises;
a) converting a compound of formula (IX) to a compound of formula (IIIb);

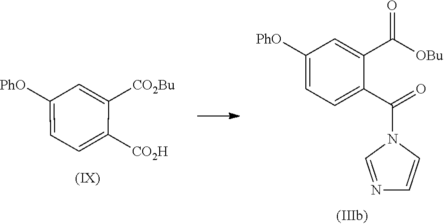

b) treating a compound of formula (IIIb) with ethyl 2-isocyanoacetate to form a compound of formula (X);

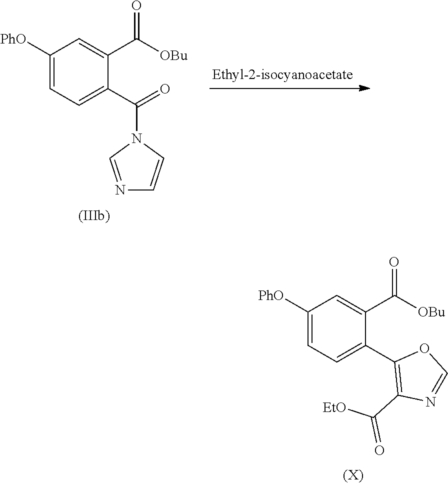

c) converting a compound of formula (X) to Roxadustat (I) or its pharmaceutically acceptable salts.

In a fifth embodiment of the present invention provides a process for the preparation of Roxadustat (I) or its pharmaceutically acceptable salts, which comprises;
a) treating a compound of formula (VII) with a methylating reagent in presence of a catalyst to form a compound of formula (VIII);

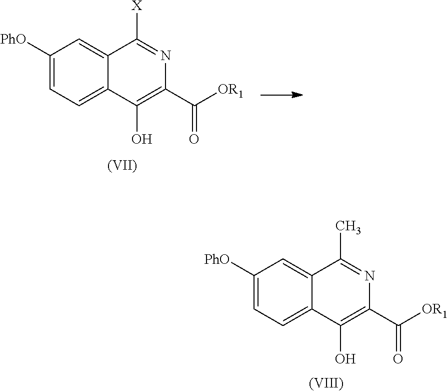

wherein R₁ is H, C₂-C₆ alkyl; X is Cl, Br, I, OTf;
b) treating a compound of formula (VIII) with glycine to form Roxadustat (I) or its pharmaceutically acceptable salts.

In a sixth embodiment of the present invention provides a process for the preparation of Roxadustat (I) or its pharmaceutically acceptable salts, which comprises;
a) treating a compound of formula (XII) with methylating agent in presence of tris(acetylacetonato)iron(III) to form a compound of formula (XIII);

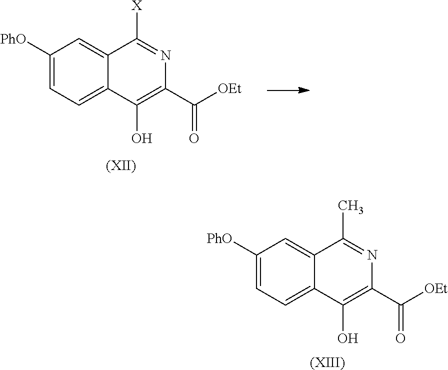

Wherein X is Cl, Br, I, OTf;
b) treating a compound of formula (XIII) with glycine to form Roxadustat (I) or its pharmaceutically acceptable salts.

In a seventh embodiment of the present invention provides a process for the preparation of Roxadustat (I) or its pharmaceutically acceptable salts, which comprises;
a) treating a compound of formula (VIII) with acid (HA) to form an acid addition salt of compound of formula (VIIIa).

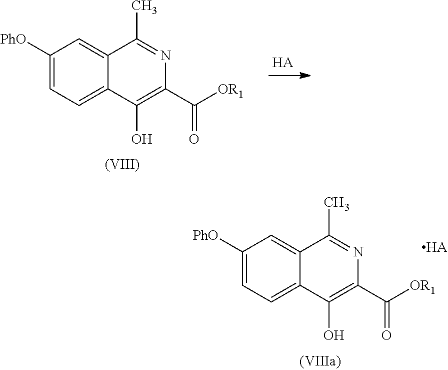

wherein R₁ is H, C₂-C₆ alkyl;
b) converting a compound of formula (VIIIa) to Roxadustat (I) or its pharmaceutically acceptable salts.

In an eighth embodiment of the present invention provides a process for the preparation of Roxadustat (I) or its pharmaceutically acceptable salts, which comprises;
c) treating a compound of formula (XIII) with acid (HA) to form an acid addition salt of compound of formula (XIIIa);

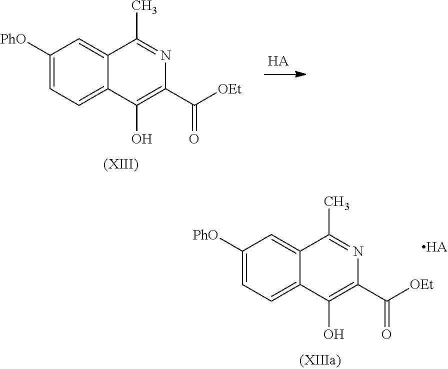

d) converting a compound of formula (XIIIa) to Roxadustat (I) or its pharmaceutically acceptable salts.

In a ninth embodiment of the present invention provides compounds of formula (III), (V), (VI), (VIII), (IX), (X), (XI), (XII), (IIIa), (IIIb), (VIIIa) and (XIIIa).

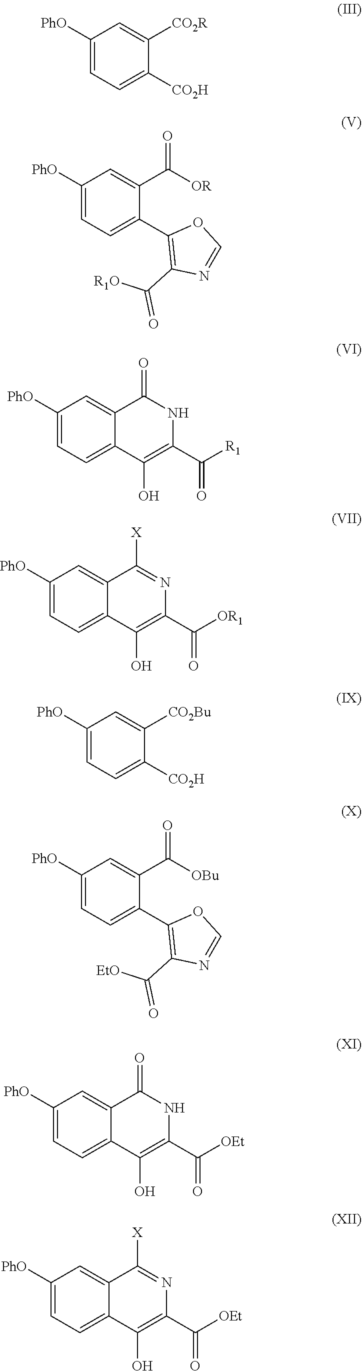

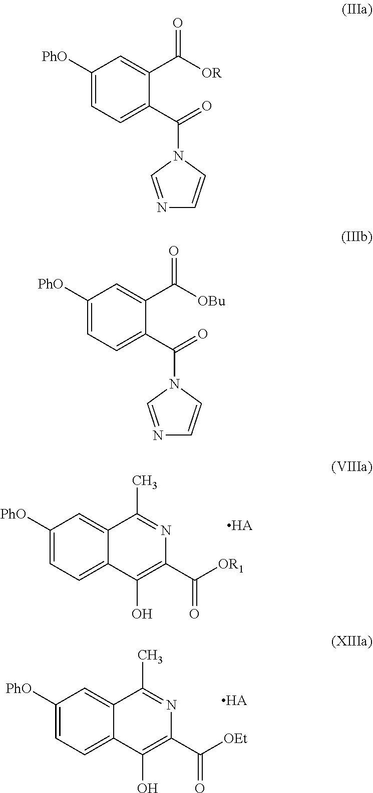

wherein R is C₁-C₆ alkyl, R₁ is H, C₂-C₆ alkyl and X is Cl, Br and I;

In a tenth embodiment of the present invention provides the use of compounds of formula (III), (V), (VI), (VIII), (IX), (X), (XI), (XII), (IIIa), (IIIb), (VIIIa) and (XIIIa) in the preparation of Roxadustat (I) or its pharmaceutically acceptable salts.

In an eleventh embodiment of the present invention provides a process for the preparation of Roxadustat (I) or its pharmaceutically acceptable salts is depicted in scheme-(IX).

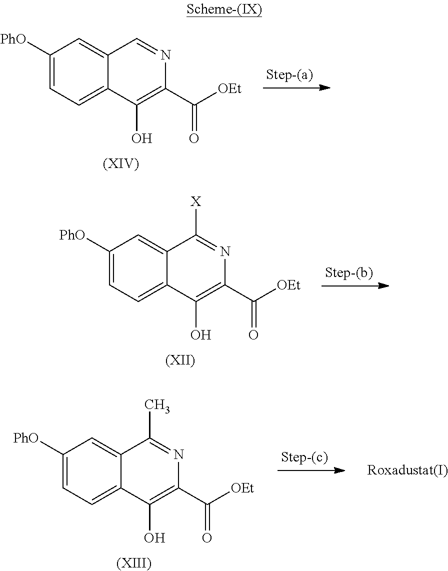

wherein X is Cl, Br and I;

In a twelfth embodiment of the present invention provides a process for the preparation of Roxadustat (I) or its pharmaceutically acceptable salts is depicted in scheme-(X).

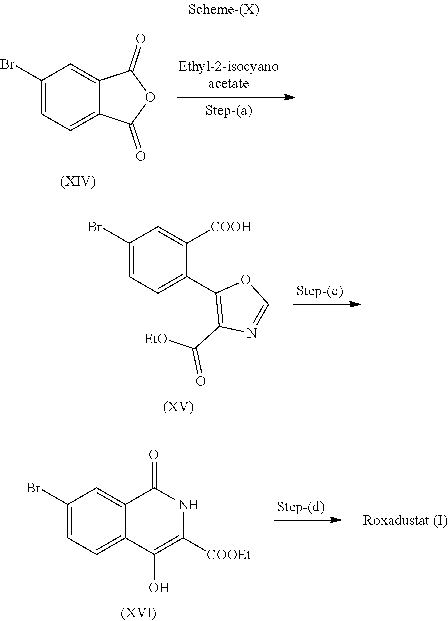

In a thirteenth embodiment of the present invention provides a process for the preparation of Roxadustat (I) or its pharmaceutically acceptable salts is depicted in scheme-(XI).

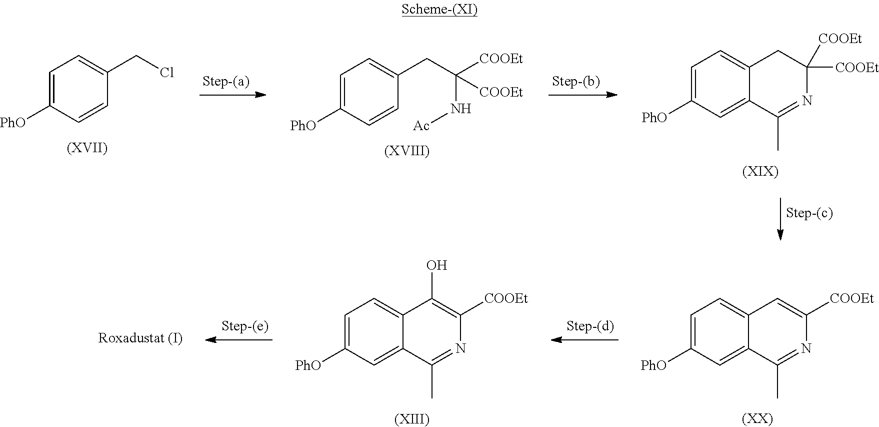

### DETAILED DESCRIPTION

The present application provides a synthetic processes for obtaining Roxadustat of formula (I) and its related intermediates.

In a first embodiment of the present invention provides a process for the preparation of Roxadustat (I) or its pharmaceutically acceptable salts is depicted in Scheme-(I).

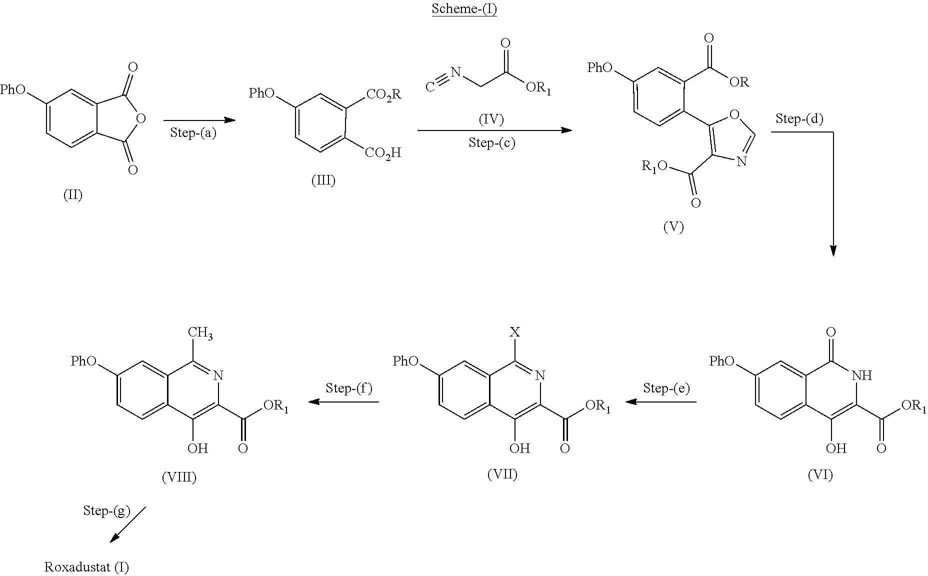

wherein R is C₁-C₆ alkyl; R₁ is H, C₂-C₆ alkyl and X is Cl, Br, I;

Suitable solvent used in step a) include, but are not limited to alcoholic solvents such as methanol, ethanol, isopropyl alcohol, n-butanol, 1-propanol or the like.

Step (b) which involves the isolation and purification of compound of formula (III) may be effected, if desired, by any suitable separation or purification procedure such as, for example, filtration, centrifugation, extraction, acid-base treatment, crystallization, conventional isolation and refining means such as concentration, concentration under reduced pressure, solvent-extraction, crystallization, phase-transfer chromatography, column chromatography.

Suitable solvent used in step b) include, but are not limited to alcoholic solvents such as methanol, ethanol, isopropyl alcohol, n-butanol, 1-propanol or the like, water, ester solvents, such as, for example, ethyl formate, methyl acetate, ethyl acetate, propyl acetate, butyl acetate, methyl propanoate, ethyl propanoate, methyl butanoate, ethyl butanoate, or the like; polar aprotic solvents such as dimethyl formamide, methyl acetamide, N-methylpyrrolidine (NMP), formamide, acetamide, propanamide, dimethyl sulfoxide or the like or mixtures thereof.

Step (c) may be carried out in the presence of one or more suitable bases. Suitable base that may be used in step (c) include, but are not limited to pyridine, piperidine, pyrimidine, triethylamine, tributylamine, N-methylmorpholine, N,N-diisopropylethylamine, diethylamine, 1,1,3,3-tetramethylguanidine, DBU, DABCO or the like.

Step (c) may be carried out in the presence of one or more suitable reagent. Suitable reagent that may be used in step c) include, but are not limited to thionyl chloride, oxalyl chloride, ethyl chloroformate, methyl chloroformate, butyl chloroformate, carbonyldiimidazole (CDI), N,N′-dicyclohexylcarbodiimide (DCC), hydroxybenzotriazole (HOBT) or the like.

Step d) may be carried out in the presence of one or more suitable acid. Suitable acid that may be used in step d) include, but are not limited to hydrochloric acid, sulphuric acid, hydrobromic acid, acetic acid, orthophosphoric acid, Lewis acid, AlCl₃, FeCl₃, bronstead acid, citric acid, oxalic acid, trifluoroacetic acid or any other suitable acids.

Suitable halogenating agent used in step e) include, but are not limited to phosphorous oxychloride, phosphorous oxybromide, chlorine, phosphorous pentachloride, thionyl chloride, liq bromine, bromine, n-bromosuccinimide (NBS), methyl iodide, methyl bromide or any other halogenating agents.

Step (f) may be carried out in the presence of one or more suitable reagents. Suitable reagents that may be used in step f) include but are not limited to triphenylphosphine palladium, trimethyl boroxine, methylmagnesium chloride, methyl magnesium bromide, methyl lithium, butyl lithium, Me₃SiX (X is Cl, Br, OTf), Tris(acetylacetonato)iron(III), iron complex, Fe(ClO4)3.9H₂O, nickel complex, copper complex, CuI, MnX₂.xH2O (X is Cl, Br, I; x is 0-4), FeCl₃, NiX₂.xH₂O (X is Cl, Br, I; x is 0-6), Ni(acac)₂, Ni(COD)₂, Cobalt complex, CoX₂(DPPH) (X is Cl, Br), CoCl₂ or mixtures thereof.

Suitable base that may be used in step (f) include, but are not limited to pyridine, piperidine, pyrimidine, triethylamine, tributylamine, N-Methyl-2-pyrrolidone (NMP), N-methylmorpholine, DBU, DABCO, sodium carbonate, potassium carbonate, sodium bicarbonate, potassium bicarbonate, sodium hydroxide, 1,1,3,3-tetramethylguanidine, potassium hydroxide, lithium hydroxide, calcium hydroxide or the like.

Compound of formula (VIII) was treated with glycine in presence of base to provide Roxadustat (I) or its pharmaceutically acceptable salts.

Suitable base that may be used in step (g) include, but are not limited to sodium methoxide, potassium methoxide, cesium methoxide, pyridine, piperidine, pyrimidine, triethylamine, tributylamine, N-methylmorpholine, DBU, DABCO sodium carbonate, potassium carbonate, sodium bicarbonate, potassium bicarbonate, 1,1,3,3-tetramethylguanidine, sodium hydroxide, potassium hydroxide, lithium hydroxide, calcium hydroxide or the like.

Step (c), step (d), step (e), step (f) and step (g) may be carried out in one or more suitable solvents. Suitable solvent that may be used in step (c) and/or step (d) and/or step (e) and/or step (f) and/or step (g) include, but are not limited to ketone solvents, such as, for example, acetone, ethyl methyl ketone, diethyl ketone, methyl isobutyl ketone, C₃-C₆ ketones or the like; aromatic hydrocarbon solvents, such as, for example, toluene, xylene, chlorobenzene, tetralin or the like; halogenated hydrocarbons such as dichloromethane, chloroform or the like; alcoholic solvents like methanol, ethanol, isopropyl alcohol, butanol or the like; aliphatic hydrocarbon solvents, such as n-pentane, n-hexane, n-heptane or the like; ether solvents, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1,4-dioxane or the like; nitrile solvent, such as, for example, acetonitrile, propionitrile, C₂-C₆ nitriles or the like; ester solvents, such as, for example, ethyl formate, methyl acetate, ethyl acetate, propyl acetate, butyl acetate, methyl propanoate, ethyl propanoate, methyl butanoate, ethyl butanoate, or the like; polar aprotic solvents such as dimethyl formamide, dimethylacetamide, N-methylpyrrolidine (NMP), formamide, acetamide, propanamide, dimethyl sulfoxide or the like; water or mixtures thereof.

The temperature at which the above steps may be carried out in between about −30° C. and about 200° C., preferably at about 0° C. and about 150° C., most preferably at about 0° C. and about 100° C., based on the solvent or mixture of solvent used in particular step.

The intermediates obtained in the present invention may be directly used for the next step with or without isolation or it may be further purified, if isolated, to improve the purity of the product.

The isolation of Roxadustat (I) may be effected, if desired, by any suitable separation or purification procedure such as, for example, filtration, centrifugation, extraction, acid-base treatment, crystallization, conventional isolation and refining means such as concentration, concentration under reduced pressure, solvent-extraction, crystallization, phase-transfer chromatography, column chromatography, or by a combination of these procedures.

In a second embodiment of the present invention provides a process for the preparation of Roxadustat (I) or its pharmaceutically acceptable salts is depicted in Scheme-(II).

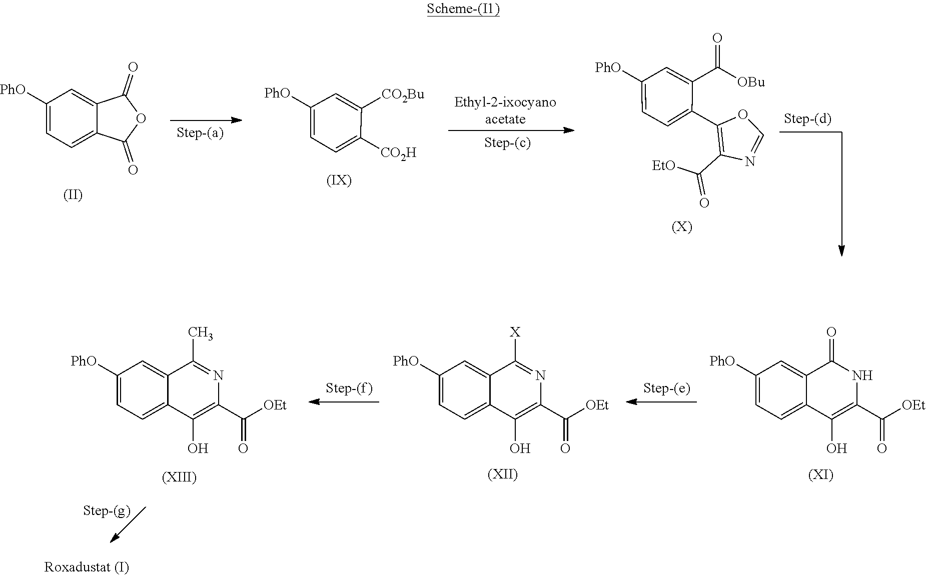

Wherein X is Cl, Br, I

The reagents, solvents and reaction conditions for steps (a) to (g) may be selected from one or more suitable reagents, solvents and process conditions as described in the steps of first embodiment of the present invention.

In a third embodiment of the present invention provides a process for the preparation of

Roxadustat (I) or its pharmaceutically acceptable salts is depicted in Scheme-III.

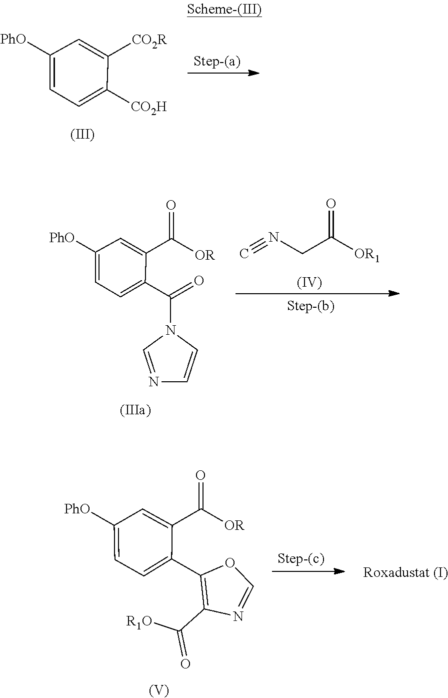

wherein R is C₁-C₆ alkyl; R₁ is C₂-C₆ alkyl.

Suitable reagent that may be used in step a) include, but are not limited to carbonyldiimidazole or the like.

Step (b) may be carried out in the presence of one or more suitable bases. Suitable base that may be used in step (b) include, but are not limited to pyridine, piperidine, pyrimidine, triethylamine, tributylamine, N-methylmorpholine, N,N-diisopropylethylamine, diethylamine, 2,2-bipyridine, 1,1,3,3-tetramethylguanidine, DBU, DABCO or the like.

Step (a) and step (b) may be carried out in one or more suitable solvents. Suitable solvent that may be used in step (a) and/or step (b) include, but are not limited to ketone solvents, such as, for example, acetone, ethyl methyl ketone, diethyl ketone, methyl isobutyl ketone, C₃-C₆ ketones or the like; aromatic hydrocarbon solvents, such as, for example, toluene, xylene, chlorobenzene, tetralin or the like; halogenated hydrocarbons such as dichloromethane, chloroform or the like; alcoholic solvents like methanol, ethanol, isopropyl alcohol, butanol or the like; aliphatic hydrocarbon solvents, such as n-pentane, n-hexane, n-heptane or the like; ether solvents, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1,4-dioxane or the like; nitrile solvent, such as, for example, acetonitrile, propionitrile, C₂-C₆ nitriles or the like; ester solvents, such as, for example, ethyl formate, methyl acetate, ethyl acetate, propyl acetate, butyl acetate, methyl propanoate, ethyl propanoate, methyl butanoate, ethyl butanoate, or the like; polar aprotic solvents such as dimethyl formamide, dimethylacetamide, N-methylpyrrolidine (NMP), formamide, acetamide, propanamide, dimethyl sulfoxide or the like; water or mixtures thereof.

Converting a compound of formula (V) to Roxadustat (I) or its pharmaceutically acceptable salts by methods known in the art.

The temperature at which the above steps may be carried out in between about −30° C. and about 200° C., preferably at about 0° C. and about 150° C., most preferably at about 0° C. and about 100° C., based on the solvent or mixture of solvent used in particular step.

The intermediates obtained in the present invention may be directly used for the next step with or without isolation or it may be further purified, if isolated, to improve the purity of the product.

In a fourth embodiment of the present invention provides a process for the preparation of Roxadustat (I) or its pharmaceutically acceptable salts is depicted in Scheme-IV.

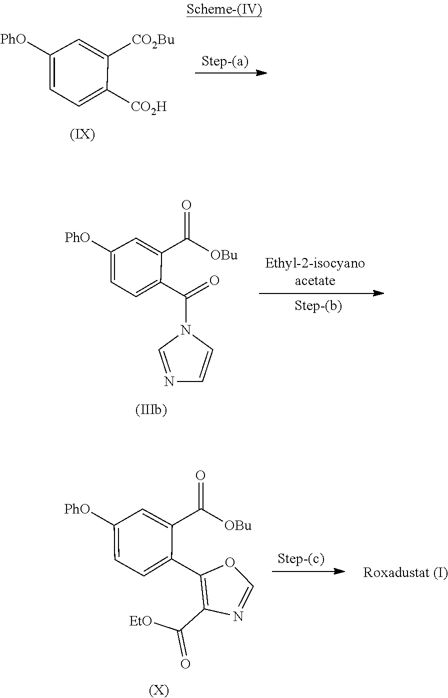

The compound of formula (IX) is treated with carbonyldiimidazole (CDI) in presence of dimethyl formamide to form a compound of formula (IIIb), followed by treating with ethyl-2-isocyanoacetate to form a compound of formula (X).

The reagents, solvents and reaction conditions for steps (a) to (c) may be selected from one or more suitable reagents, solvents and process conditions as described in the steps of third embodiment of the present invention.

In a fifth embodiment of the present invention provides a process for the preparation of Roxadustat (I) or its pharmaceutically acceptable salts is depicted in Scheme-V.

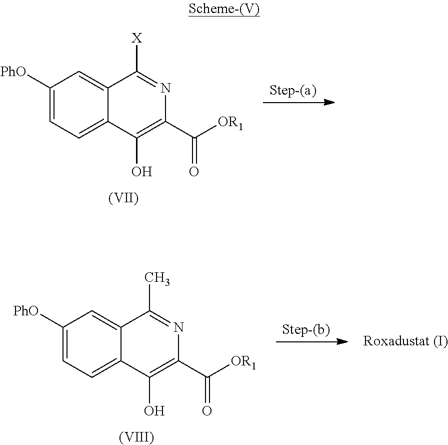

wherein R₁ is H, C₂-C₆ alkyl; X is Cl, Br, I, OTf;

Suitable methylating agents that may be used in step a) include, but are not limited to trimethyl boroxine, methylmagnesium chloride, methyl magnesium bromide, methyl lithium, trimethyl silyl halides, methyl iodide, dimethyl sulfate or any other methylating agents.

Catalyst that may be used in step a) include, but are not limited to triphenylphosphine palladium, Tris(acetylacetonato)iron(III), iron complex, Fe(ClO₄)₃.9H₂O, nickel complex, copper complex, CuI, MnX₂.xH₂O (X is Cl, Br, I; x is 0-4), FeCl₃, NiX₂.xH₂O (X is Cl, Br, I; x is 0-6), Ni(acac)₂, Ni(COD)₂, Cobalt complex, CoX₂(DPPH) (X is Cl, Br), CoCl₂ or any other catalysts.

Suitable base that may be used in step (a) include, but are not limited to pyridine, piperidine, pyrimidine, triethylamine, tributylamine, N-Methyl-2-pyrrolidone (NMP), N-methylmorpholine, DBU, DABCO sodium carbonate, potassium carbonate, sodium bicarbonate, potassium bicarbonate, sodium hydroxide, potassium hydroxide, lithium hydroxide, calcium hydroxide or the like.

Step (a) may be carried out in one or more suitable solvents. Suitable solvent that may be used in step (a) include, but are not limited to ketone solvents, such as, for example, acetone, ethyl methyl ketone, diethyl ketone, methyl isobutyl ketone, C₃-C₆ ketones and the like; aromatic hydrocarbon solvents, such as, for example, toluene, xylene, chlorobenzene, tetralin, and the like; halogenated hydrocarbons such as dichloromethane, chloroform, carbon tetrachloride and the like; alcoholic solvents like methanol, ethanol, isopropyl alcohol and the like; aliphatic hydrocarbon solvents, such as n-pentane, n-hexane, n-heptane and the like; ether solvents, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1,4-dioxane, and the like; nitrile solvent, such as, for example, acetonitrile, propionitrile, C₂-C₆ nitriles and the like; ester solvents, such as, for example, ethyl formate, methyl acetate, ethyl acetate, propyl acetate, butyl acetate, methyl propanoate, ethyl propanoate, methyl butanoate, ethyl butanoate, or the like; polar aprotic solvents such as dimethyl formamide, dimethylacetamide, N-methylpyrrolidine (NMP), formamide, acetamide, propanamide, dimethyl sulfoxide and the like; water or mixtures thereof.

Compound of formula (VIII) was treated with glycine in presence of base to provide Roxadustat (I) or its pharmaceutically acceptable salts by methods known in the art.

The temperature at which the above steps may be carried out in between about −60° C. and about 200° C., preferably at about −60° C. and about 150° C., most preferably at about −30° C. and about 100° C., based on the solvent or mixture of solvent used in particular step.

In a sixth embodiment of the present invention provides a process for the preparation of Roxadustat (I) or its pharmaceutically acceptable salts is depicted in Scheme-VI.

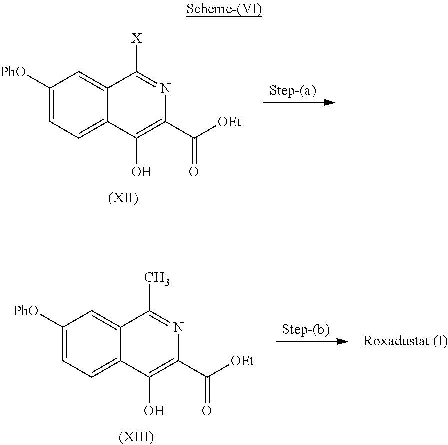

wherein X is Cl, Br, I, OTf;

Compound of formula (XII) is treated with tris(acetylacetonato)iron(III) in presence of tetrahydrofuran and n-methyl-pyrrolidine (NMP), methyl magnesium chloride/methyl magnesium bromide to form a compound of formula (XIII). Compound of formula (XIII) was treated with glycine in presence of base to provide Roxadustat (I) or its pharmaceutically acceptable salts by methods known in the art.

The reagents, solvents and reaction conditions for steps (a) and (b) may be selected from one or more suitable reagents, solvents and process conditions as described in the steps of fifth embodiment of the present invention.

In a seventh embodiment of the present invention provides a process for the preparation of Roxadustat (I) or its pharmaceutically acceptable salts is depicted in Scheme-VII.

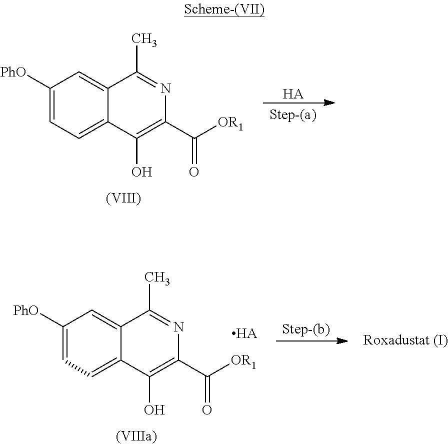

wherein R₁ is H, C₂-C₆ alkyl;

Suitable acid that may be used in step (a) include, but are not limited to: hydrochloric acid, acetic acid, sulfuric acid, p-toluene sulfonic acid, oxalic acid, trifluoroacetic acid or any other suitable acid.

Step (a) may be carried out in one or more suitable solvents. Suitable solvent that may be used in step (a) include, but are not limited to ketone solvents, such as, for example, acetone, ethyl methyl ketone, diethyl ketone, methyl isobutyl ketone, C₃-C₆ ketones and the like; aromatic hydrocarbon solvents, such as, for example, toluene, xylene, chlorobenzene, tetralin, and the like; halogenated hydrocarbons such as dichloromethane, chloroform, carbon tetrachloride and the like; alcoholic solvents like methanol, ethanol, isopropyl alcohol and the like; aliphatic hydrocarbon solvents, such as n-pentane, n-hexane, n-heptane and the like; ether solvents, such as, for example, diethyl ether, diisopropyl ether, tert-butyl methyl ether, dibutyl ether, tetrahydrofuran, 1,2-dimethoxyethane, 2-methoxyethanol, 2-ethoxyethanol, anisole, 1,4-dioxane, and the like; nitrile solvent, such as, for example, acetonitrile, propionitrile, C₂-C₆ nitriles and the like; ester solvents, such as, for example, ethyl formate, methyl acetate, ethyl acetate, propyl acetate, butyl acetate, methyl propanoate, ethyl propanoate, methyl butanoate, ethyl butanoate, or the like; polar aprotic solvents such as dimethyl formamide, dimethylacetamide, N-methylpyrrolidine (NMP), formamide, acetamide, propanamide, dimethyl sulfoxide and the like; water or mixtures thereof.

Compound of formula (VIIIa) was treated with glycine in presence of base to provide Roxadustat (I) or its pharmaceutically acceptable salts by methods known in the art.

The temperature at which the above steps may be carried out in between about 0° C. and about 100° C., preferably at about 0° C. and about 80° C., most preferably at about 10° C. and about 50° C., based on the solvent or mixture of solvent used in particular step.

In an eighth embodiment of the present invention provides a process for the preparation of Roxadustat (I) or its pharmaceutically acceptable salts is depicted in scheme-(VIII).

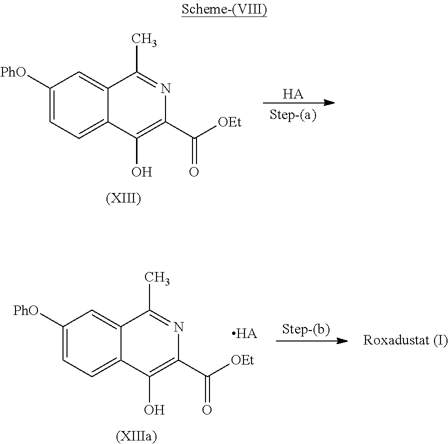

The reagents, solvents and reaction conditions for steps (a) to (c) may be selected from one or more suitable reagents, solvents and process conditions as described in the steps of seventh embodiment of the present invention.

In a ninth embodiment of the present invention provides compounds of formula (III), (V), (VI), (VIII), (IX), (X), (XI), (XII), (IIIa), (IIIb), (VIIIa) and (XIIIa).

In a tenth embodiment of the present invention provides the use of compounds of formula (III), (V), (VI), (VIII), (IX), (X), (XI), (XII), (IIIa), (IIIb), (VIIIa) and (XIIIa) in the preparation of Roxadustat (I) or its pharmaceutically acceptable salts.

In an eleventh embodiment of the present invention provides a process for the preparation of Roxadustat (I) or its pharmaceutically acceptable salts is depicted in scheme-IX.

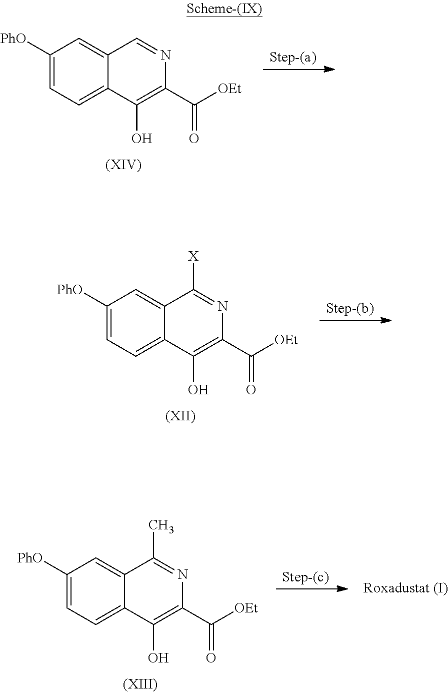

wherein X is Cl, Br and I;

Suitable halogenating agent may be used in step a) include, but are not limited to phosphorus tribromide, aluminum tribromide, N-bromosuccinimide (NBS), N-chloro succinimide, bromine, chloridne, phosphorous trichloride, phosphorous pentachloride, phosphorous pentabromide or any other halogenating agent.

Compound of formula (XII) is reacted with tetrakis triphenylphosphine palladium and trimethyl boroxine in presence of base to form a compound of formula (XIII).

Suitable base that may be used in step (b) include, but are not limited to pyridine, piperidine, pyrimidine, triethylamine, tributylamine, N-methylmorpholine, DBU, DABCO sodium carbonate, potassium carbonate, sodium bicarbonate, potassium bicarbonate, sodium hydroxide, potassium hydroxide, lithium hydroxide, calcium hydroxide or the like.

Compound of formula (XIII) was treated with glycine in presence of base to provide Roxadustat (I) or its pharmaceutically acceptable salts.

The temperature at which the above steps may be carried out in between about 0° C. and about 200° C., preferably at about 0° C. and about 150° C., most preferably at about 0° C. and about 100° C., based on the solvent or mixture of solvent used in particular step.

In a twelfth embodiment of the present invention provides a process for the preparation of Roxadustat (I) or its pharmaceutically acceptable salts is depicted in scheme-(X).

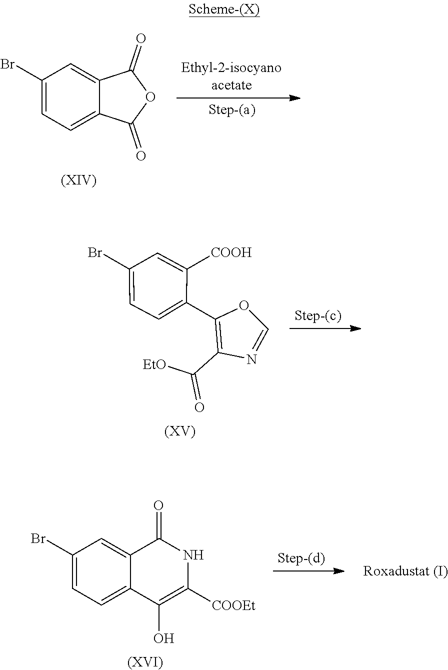

Step (a) may be carried out in the presence of one or more suitable bases. Suitable base that may be used in step (a) include, but are not limited to pyridine, piperidine, pyrimidine, triethylamine, tributylamine, N-methylmorpholine, N,N-diisopropylethylamine, diethylamine, 1,1,3,3-tetramethylguanidine, DBU, DABCO or the like

Step (b) which involves the isolation and purification of compound of formula (XI) may be effected, if desired, by any suitable separation or purification procedure such as, for example, filtration, centrifugation, extraction, acid-base treatment, crystallization, conventional isolation and refining means such as concentration, concentration under reduced pressure, solvent-extraction, crystallization, phase-transfer chromatography, column chromatography, or by a combination of these procedures.

Suitable solvent used in step b) include, but are not limited to alcoholic solvents such as methanol, ethanol, isopropyl alcohol, n-butanol, 1-propanol or the like, water, ester solvents, such as, for example, ethyl formate, methyl acetate, ethyl acetate, propyl acetate, butyl acetate, methyl propanoate, ethyl propanoate, methyl butanoate, ethyl butanoate, or the like; polar aprotic solvents such as dimethyl formamide, methyl acetamide, N-methylpyrrolidine (NMP), formamide, acetamide, propanamide, dimethyl sulfoxide or the like or mixtures thereof.

Step c) may be carried out in the presence of one or more suitable acid. Suitable acid that may be used in step d) include, but are not limited to hydrochloric acid, sulphuric acid, hydrobromic acid, acetic acid or any other suitable acids.

The compound of formula (XVI) may be converted to Roxadustat (I) or its pharmaceutically acceptable salts by methods known in the literature.

The temperature at which the above steps may be carried out in between about 0° C. and about 200° C., preferably at about 0° C. and about 150° C., most preferably at about 0° C. and about 100° C., based on the solvent or mixture of solvent used in particular step.

In a thirteenth embodiment of the present invention provides a process for the preparation of Roxadustat (I) or its pharmaceutically acceptable salts is depicted in scheme-(XI).

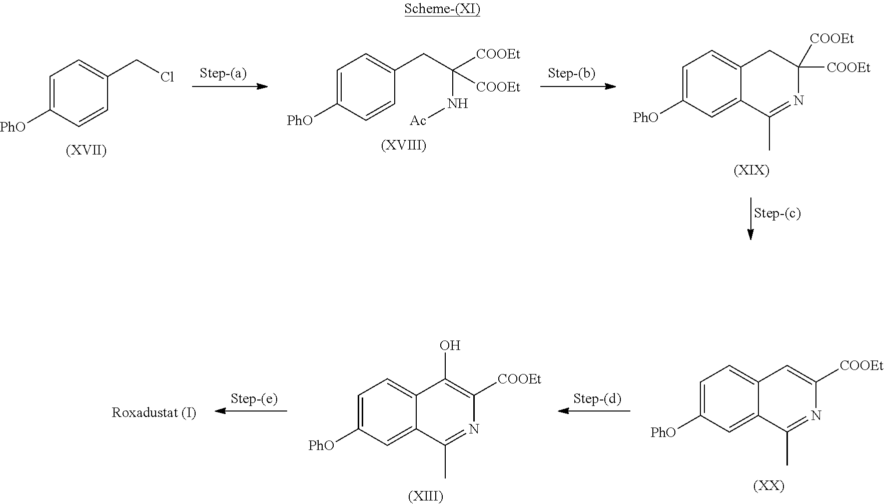

Step (a) may be carried out in the presence of one or more suitable bases. Suitable base that may be used in step (a) include, but are not limited to pyridine, piperidine, pyrimidine, triethylamine, tributylamine, N-methylmorpholine, N,N-diisopropylethylamine, diethylamine, 1,1,3,3-tetramethylguanidine, DBU, DABCO and the like; sodium carbonate, cesium carbonate, potassium carbonate, sodium bicarbonate, potassium bicarbonate, potassium iodide, metal hydroxide like sodium hydroxide, potassium hydroxide, lithium hydroxide, calcium hydroxide and magnesium hydroxide or mixtures thereof.

Suitable reagent that may be used in step b) include, but are not limited to phosphorous oxychloride, phosphorous oxybromide or any other halogenating agent.

Lithium salt may be used in step c) include, but are not limited to lithium chloride, lithium bromide, lithium iodide. Suitable base that may be used in step (c) and step (d) include, but are not limited to pyridine, piperidine, pyrimidine, triethylamine, tributylamine, N-methylmorpholine, N,N-diisopropylethylamine, diethylamine, 1,1,3,3-tetramethylguanidine, DBU, DABCO, sodium carbonate, cesium carbonate, potassium carbonate, sodium bicarbonate, potassium bicarbonate, sodium hydroxide, potassium hydroxide, lithium hydroxide, calcium hydroxide and magnesium hydroxide or mixtures thereof.

The temperature at which the above steps may be carried out in between about 0° C. and about 200° C., preferably at about 0° C. and about 150° C., most preferably at about 0° C. and about 100° C., based on the solvent or mixture of solvent used in particular step.

In each stage the compounds of all embodiments of the present application are isolated from the reaction mixture may involve methods including removal of solvent, cooling, crash cooling, concentrating the mass, evaporation, flash evaporation, simple evaporation, fast solvent evaporation, rotational drying, spray drying, thin-film drying, agitated thin film drying, agitated nutsche filter drying, pressure nutsche filter drying, freeze-drying, rotary vacuum paddle dryer, adding anti-solvent or the like. Stirring or other alternate methods such as shaking, agitation, or the like, may also be employed for the isolation.

The processes of the present invention is easy to handle, environment friendly, provides better yield with required purity and it may also be practiced at on industrial scale.

Certain specific aspects and embodiments of the present invention will be explained in more detail with reference to the following examples, which are provided for purposes of illustration only and should not be construed as limiting the scope of the present invention in any manner.

### EXAMPLES

### Example-1

### Preparation of 2-(methoxycarbonyl)-4-phenoxybenzoic acid

4-Phenoxyphthalic acid (50 g), acetic acid (525 mL) and acetic anhydride (546 mL) were charged in RBF at 28° C. The reaction mixture was heated to 120° C. and maintained for 3-4 hours. The reaction mixture was cooled to 28° C. Methanol (250 mL) was added to the reaction mass at 28° C. and stirred for 3 hours. Charcoal (5 g) was added to the reaction mass and stiffed for 10 minutes. The solvent from the reaction mass was distilled under vacuum. n-Heptane (250 mL) was added to the reaction mass at 28° C. Filtered the reaction mass and washed with n-heptane (100 mL), dried at 55° C. for 5-6 hours to give the title compound. Yield: 80.63%

### Example-2

### Purification of 2-(methoxycarbonyl)-4-phenoxybenzoic acid

Mixture of 2-(methoxycarbonyl)-4-phenoxybenzoic acid and 2-(methoxycarbonyl)-5-phenoxybenzoic acid (5 g), methanol (45 mL) and water (5 mL) were charged in RBF at 27° C. and stiffed for 5 minutes. The reaction mixture was heated to 65° C. and maintained for 60 minutes. The reaction mass was cooled to 27° C. and maintained for 4 hours. Filtered the reaction mass and washed with methanol (5 mL). The wet product, methanol (13.5 mL), water (1.5 mL) were again charged into RBF at 27° C. and stirred for 5 minutes. The reaction mixture was heated to 60° C. and maintained for 30 minutes. The reaction mass was cooled to 27° C. and maintained for 3-4 hours. The obtained solid was filtered and washed with methanol (1.5 mL), dried at 57° C. for 4 hours to give the title compound.

### Example-3

### Preparation of ethyl 5-(2-(methoxycarbonyl)-4-phenoxyphenyl) oxazole-4-carboxylate

2-(Methoxycarbonyl)-4-phenoxybenzoic acid (0.2 g), dichloromethane (1 mL), oxalyl chloride (0.653 g), DMF (10 μL) were charged in RBF at 27° C. and maintained for 60 minutes. The reaction mixture was heated to 50° C. The solvent from the reaction mass was completely evaporated under vacuum. THF (0.2 mL) was added to the reaction mass 28° C. The resultant reaction mass was added to the solution containing ethyl 2-isocyanoacetate (0.1 g), triethylamine (0.245 g) and THF (0.2 mL) at 0° C. The reaction mass was heated to 25-30° C. and maintained for 60 minutes. The reaction mass was further heated to 65° C. and maintained for two hours. Water (0.1 mL) and ethyl aceate (0.1 mL) were added to the reaction mass at 27° C. and the layers were separated. The organic layer was distilled at 50° C. completely to give the title compound. Yield: 37.17

### Example-4

### Preparation of ethyl 4-hydroxy-1-oxo-7-phenoxy-1,2-dihydroisoquinoline-3-carboxylate

Ethyl 5-(2-(methoxycarbonyl)-4-phenoxyphenyl)oxazole-4-carboxylate (0.05 g), methanol (0.25 mL) and conc. HCl (0.050 mL) were charged at 28° C. The temperature of the reaction was raised to 55-60° C. and maintained for 4-5 hours. The reaction mass was cooled to 25-35° C. and stirred for 2 hours. The obtained solid was filtered and washed with methanol (0.05 mL), dried at 50° C. for 60 minutes to give the title compound. Yield: 25%

### Example-5

### Preparation of ethyl 1-chloro-4-hydroxy-7-phenoxyisoquinoline-3-carboxylate

Ethyl 4-hydroxy-1-oxo-7-phenoxy-1,2-dihydroisoquinoline-3-carboxylate (6.3 g), POCl₃ (63 mL) were charged at 28° C. The reaction mixture was heated to 90° C. and maintained for 3-4 hours. The reaction mass was cooled to 27° C. and stiffed for 30 minutes. The reaction mass was charged to water (400 mL) and stirred for 10-20 minutes. The obtained solid was filtered and washed with water (31.5 mL), dried at 50° C. for 3-4 hours to give the title compound. Yield: 75.19%

### Example-6

### Preparation of ethyl 4-hydroxy-1-methyl-7-phenoxyisoquinoline-3-carboxylate

Ethyl-1-chloro-4-hydroxy-7-phenoxyisoquinoline-3-carboxylate (1.5 g), dioxane (15 mL) and tetrakis triphenyl phosphine palladium (0.555 g) were charged at 28° C. and stirred for 5 minutes. Trimethyl boroxine (0.822 g) was added to the reaction mixture at 28° C. The reaction mixture was heated to 80° C. and maintained for 4 hours. The reaction mass was cooled to 28° C. Water (7.5 mL) and ethyl acetate (15 mL) were added to the reaction mass and the layers were separated. The solvent from the organic layer was distilled at 50° C. to obtain crude compound. The crude compound was charged in 20% ethyl acetate in hexane (20 mL) and stiffed for 5 minutes. The solvent from the reaction mass was concentrated under vacuum at 50° C. to give the title compound.

### Example-7

### Preparation of Roxadustat

Ethyl-4-hydroxy-1-methyl-7-phenoxyisoquinoline-3-carboxylate (1.2 g), sodium methoxide (54 g), methanol (6 mL) and glycine (75.07 g) were charged at 28° C. and stiffed for 5 minutes. The reaction mixture was heated to 100° C. and maintained for 12-14 hours. The reaction mixture was cooled to 28° C. The solvent from reaction mixture was concentrated under vacuum at 28° C. Water (12 mL) and ethyl acetate (12 mL) were charged to the reaction mass at 28° C. and layers were separated. The aqueous layer was washed with ethyl acetate (6 mL). The aqueous layer was slowly adjusted the pH 3-3.5 by using acetic acid (3.6 mL). The obtained solid was filtered and washed with water (6 mL), dried at 50° C. for 2 hours. The obtained product was slurried in acetone (6 mL) and stirred for 20 minutes, filtered the solid and washed with acetone (6 mL) to give the title compound.

### Example-8

### Preparation of 2-(butoxycarbonyl)-4-phenoxybenzoic acid

4-Phenoxyphthalic acid (258.2 g), acetic acid (105 mL) and acetic anhydride (102.09 mL) were charged in RBF at 28° C. The reaction mixture was heated to 120° C. and maintained for 3-4 hours. The reaction mixture was concentrated at 65° C. under vacuum. The reaction mixture was cooled to 28° C. n-Butanol (500 mL) was added to the reaction mass at 28° C. and stirred for 5-6 hours. Charcoal (5 g) was added to the reaction mass and stirred for 10 minutes. The obtained solid was filtered and washed with n-butanol (250 mL), dried at 65° C. for 4-5 hours to give the title compound.

NMR data: 0.88-0.86 (3H, triplet), 1.36-1.29 (2H, multiplet), 1.61-1.57 (2H, multiplet), 4.14-4.12 (2H, triplet), 6.89 (1H, Singlet), 7.04-7.03 (1H doublet), 7.10-7.08 (2H, doublet), 7.22-7.20 (1H, doublet), 7.45-7.42 (2H, triplet), 7.77-7.76 (1H, doublet). Mass m/z: 313.01 (M−1).

### Example-9

### Ethyl-5 -(2-(butoxycarbonyl)-4-phenoxyphenyl)oxazole-4-carboxylate

2-(Butoxycarbonyl)-4-phenoxybenzoic acid (0.5 g), dichloromethane (12.5 mL), oxalyl chloride (3.03 g), DMF (0.050 mL) were charged in RBF at 27° C. and maintained for 1-2 hours. The reaction mixture was heated to 50° C. The solvent from the reaction mass was completely evaporated under vacuum. THF (1 mL) was added to the reaction mass 28° C. The solution of ethyl 2-isocyanoacetate (0.216 g), triethylamine (0.530 g) and THF (1 mL) was added to the reaction mass at 28° C. and maintained for 1-2 hours. Water (0.5 mL) and ethyl acetate (0.5 mL) were added to the reaction mass at 27° C. and the layers were separated. The organic layer was distilled at 50° C. completely to give the title compound.

### Example-10

### Preparation of ethyl 4-hydroxy-1-oxo-7-phenoxy-1,2-dihydro isoquinoline-3-carboxylate

Ethyl 5-(2-(butoxycarbonyl)-4-phenoxyphenyl)oxazole-4-carboxylate (1 g), methanol (7 mL) and conc. HCl (1.4 mL) were charged at 28° C. The temperature of the reaction was raised to 60° C. and maintained for 8-9 hours. The reaction mass was cooled to 28° C. and stirred for 60 minutes. The solvent from the reaction mass was concentrated under vacuum at 50° C. Methanol (2.1 mL) was added to the reaction mass at 27° C. The obtained solid was filtered and washed with methanol (0.7 mL), dried at 50° C. for 1-2 hours to give the title compound.

### Example-11

### Preparation of 5-bromo-2-(4-(ethoxycarbonyl)oxazol-5-yl)benzoic acid

DBU (33.5 gm) and THF (250 ml) were charged at 28° C. and stirred for 5 minutes. The solution of 5-bromoisobenzofuran-1,3-dione (50 gm), ethyl 2-isocyanoacetate (27.4 gm) and THF (500 ml) was added to the reaction mixture slowly at room temperature and maintained for about 3 hours. Quench the reaction mixture with DM water (250 ml) and concentrate the solvent under vacuum. Conc. HCl (25 ml) and DM water (250 ml) were added to the reaction mixture to adjust the pH. Ethylacetate (500 ml) was charged and stirred for 15 minutes at room temperature. The layers were separated and the organic layer was washed with DM water (250 ml) and concentrate the solvent under vacuum. Isopropyl alcohol (250 ml) was charged into the flask and maintained for about 15 hours at room temperature. Filtered the solid and washed the solid with isopropyl alcohol (50 ml) and dried under vacuum at 55° C. to give the title compound.

### Example-12

### Purification of 5-bromo-2-(4-(ethoxycarbonyl)oxazol-5-yl)benzoic acid

5-bromo-2-(4-(ethoxycarbonyl)oxazol-5-yl)benzoic acid (5 gm), isopropyl alcohol (50 ml) were charged into a round bottom flask and heated to 50° C. The reaction mass was cooled to room temperature and maintained overnight and filtered the solid under vacuum for about 4 hours at 55° C. to give the title compound.

### Example-13

### Preparation of ethyl 7-bromo-4-hydroxy-1-oxo-1,2-dihydroisoquinoline-3-carboxylate

5-Bromo-2-(4-(ethoxycarbonyl)oxazol-5-yl)benzoic acid (5 g), conc. HCl (5 mL) and methanol (25 mL) were charged at 28° C. and stirred for 5 minutes. The reaction mixture temperature was raised to 50° C. and maintained for 3-4 hours. The reaction mass was cooled to 28° C. and stirred for 30 minutes. Filtered the solid and washed with methanol (5 mL). Methanol (25 mL) was added to the obtained solid and heated to 55° C. and maintained for 60 minutes. Filtered the solid and washed with methanol (5 mL), dried at 50° C. under vacuum to give the title compound.

### Example-14

### Preparation of diethyl 2-acetamido-2-(4-phenoxybenzyl)malonate

1-(Chloromethyl)-4-phenoxybenzene (40 g), diethyl 2-acetamidomalonate (43.7 g) and acetonitrile (400 mL) were charged in to the RBF at 27° C. Potassium carbonate (50.6) and potassium iodide (30.4) were added to the reaction mixture at 27° C. The reaction mixture was heated to 90° C. and maintained for 5-6 hours. The reaction mass was concentrated under vacuum at 54° C. Ethyl acetate (400 mL) and water (400 mL) were added to the reaction mass at 28° C. and layers were separated. The aqueous layer was extracted with ethyl acetate (200 mL). Combined the organic layers and organic layer was washed with water (400 mL) and 10% NaCl solution (400 mL) and layers were separated. The solvent from the organic layer was concentrated under vacuum at 52° C. and chased with n-heptane (200 mL). Ethyl aceate (40 mL) was added to the reaction mass and heated to 52° C., maintained for 30 minutes. N-Heptane (120 mL) was added to the reaction mass and heated to 52° C., maintained for 30-60 minutes. The reaction mass was cooled to 26° C. The obtained solid was filtered and washed with n-heptane (40 mL), dried at 55° C. for 10-12 hours to give title compound.

### Example-15

### Preparation of diethyl 1-methyl-7-phenoxyisoquinoline-3,3 (4H)-dicarboxylate

Diethyl 2-acetamido-2-(4-phenoxybenzyl)malonate (25 g) and POCl₃(250 mL) were charged in RBF at 28° C. The reaction mixture was heated to 103° C. and maintained for 4 hours. The reaction mixture was cooled to 50° C. and was concentrated under vacuum at 50° C. Ethyl acetate (1000 mL) 20% sodium carbonate solution were added to the reaction mass at 2° C. and layers were separated. Organic layer was washed with brine solution and was concentrated under vacuum at 50° C. Further, the obtained crude product was purified by flash chromatography to give the title compound.

### Example-16

### Preparation of ethyl 1-methyl-7-phenoxyisoquinoline-3-carboxylate

Diethyl 1-methyl-7-phenoxyisoquinoline-3,3(4H)-dicarboxylate (1 g), LiCl (0.167 g), DMSO (10 mL) were charged in to RBF at 27° C. and stirred for 10 minutes. The reaction mixture was heated to 150° C. and maintained for 6 hours. The reaction mixture was cooled to 95° C. and maintained for 12 hours. Ethyl acetate (30 mL) and sodium bicarbonate solution were added to the reaction mass at 27° C. and layers were separated.

The organic layer was washed with sodium chloride solution and the organic layer was concentrated at 50° C. The obtained crude product was purified by column chromatography to give the title compound.

### Example-17

### Preparation of ethyl-4-hydroxy-1-methyl-7-phenoxyisoquinoline-3-carboxylate

Ethyl-1-methyl-7-phenoxyisoquinoline-3-carboxylate (0.2 g) and glacial acetic acid (0.195 g) were charged at 27° C. and stiffed for 10 minutes. 30% hydrogen peroxide (0.066 g) was added to the reaction mixture at 27° C. and stiffed for 5 minutes. The reaction mixture was heated to 70° C. 30% hydrogen peroxide (0.044 g) and glacial acetic acid (0.156) were slowly added to the reaction mass at 70° C. and maintained for 7-10 hours. The reaction mass was cooled to 50° C. The reaction mass was concentrated at 50° C. and chased with ethanol (2×0.5 mL), distilled completely under vacuum. Dichloromethane (25 mL) and 5% sodium bicarbonate solution (0.05 g in 0.5 mL) were added to the reaction mass and layers were separated. The organic layer was dried with sodium sulfate (1 g). P-toluene sulfonyl chloride (0.248 g) was added to the organic layer and heated to 38° C., maintained for 3-4 hours. The solvent from the reaction mass was completely distilled at 45° C. and chased with methanol and stirred for 10 minutes. Filtered the solid and dried at 50° C. to give the title compound.

### Example-18

### Preparation of ethyl-1-bromo-4-hydroxy-7-phenoxyisoquinoline-3-carboxylate

Ethyl-4-hydroxy-7-phenoxyisoquinoline-3-carboxylate (5 g), N-Bromosuccinamide (3.02 g), Benzoyl peroxide (0.196 g) and carbon tetrachloride (50 mL) were charged at 26° C. and stirred for 10 minutes. The reaction mixture was heated to 80° C. and maintained for 6-7 hours. The reaction mass was distilled completely at 50° C. under vacuum. Ethyl acetate (15 mL) and water (15 mL) were added to the above crude and stiffed for 20 minutes. Layers were separated and the organic layer was washed with water (2×10 mL). The solvent from the organic layer was concentrated at 40° C. under vacuum. Dichloromethane (0.6 mL) and hexane (3 mL) were added to the above crude at 26° C. and maintained for 60 minutes. Filtered the solid and washed with hexane (3 mL), dried at 45° C. for 3 hours to give the title compound.

### Example-19

### Preparation of ethyl 4-hydroxy-1-methyl-7-phenoxyisoquinoline-3-carboxylate

Ethyl-1-bromo-4-hydroxy-7-phenoxyisoquinoline-3-carboxylate (2 g), dioxane (20 mL), tetrakis triphenyl phosphine palladium (0.655 g) and potassium carbonate (2.133 g) were charged at 28° C. and stirred for 5 minutes. Trimethyl boroxine (0.970 g) was slowly added to the reaction mixture at 28° C. The reaction mixture was heated to 80° C. and maintained for 4 hours. The reaction mass was cooled to 28° C. Water (10 mL) and ethyl acetate (20 mL) were added to the reaction mass and the layers were separated. The solvent from the organic layer was distilled at 50° C. to obtain crude compound. Methanol (10 mL) was added to the above obtained crude and maintained for 3 hours. Filtered the solid and washed with methanol (2 mL), dried at 50° C. for 5 hours to give the title compound.

### Example-20

### Preparation of ethyl 5-(2-(butoxycarbonyl)-4-phenoxyphenyboxazole-4-carboxylate

2-(Butoxycarbonyl)-4-phenoxybenzoic acid (1.0 g) and DMF (3 mL) were charged in RBF at 27° C. and stirred for 5-10 minutes. Di(1H-imidazol-1-yl)methanone (0.645 g) was added at 27° C. to the reaction mixture and stirred for 5-10 minutes. The solution of ethyl-2-isocyanoacetate (0.540 g), triethylamine (0.966 g) and DMF (2 mL) was added to the reaction mass at 27° C. and stirred for 10-20 minutes. The reaction mixture was heated to 74° C. and maintained for 10 hours. Water (10 mL) and ethyl acetate (20 mL) were added to the reaction mass at 28° C. and the layers were separated. The aqueous layer was washed with ethyl acetate (10 mL). Combine the organic layer was distilled at 50° C. completely under vacuum, followed by purified the crude through column chromatography to give the title compound. Yield: 69.1%

### Example-21

### Preparation of ethyl 5-(2-(butoxycarbonyl)-4-phenoxyphenyl)oxazole-4-carboxylate

2-(Butoxycarbonyl)-4-phenoxybenzoic acid (1200 g) and THF (6 mL) were charged in reactor and stiffed for 5-10 minutes. Di(1H-imidazol-1-yl) methanone (929 g) was added to the reaction mixture and stirred for 5-10 minutes. The reaction mass was heated to 45-50° C. and maintained for 3-4 hours at 45-50° C. The reaction mass was cooled to 5-10° C. Ethyl-2-isocyanoacetate (648 g) was added to the reaction mass and stirred for 5-10 minutes. DBU (1162 g) was slowly added to the reaction mass and stirred for 5-10 minutes. The reaction mass was heated to 20-30° C. and maintained for 12 hours. Toluene (3 L) and DM-water (6 L) were charged in to the reaction mass and stirred for 5-10 minutes. Layers were separated and the aqueous layer extracted with toluene (3 L) and stirred for 5-10 minutes. Combine the organic layer and washed with DM-water (2×4 L). Organic layer was distilled at below 60° C. completely under vacuum. The reaction mass was cooled to 25-30° C. Isopropyl alcohol (2 L) was added to the reaction mass and distilled at below 60° C. Isopropyl alcohol (4.8 L) was added to the reaction mass and cooled to below 30° C. Hydrochloric acid (32%; 1.3 L) was slowly added to the reaction mass at below 30° C. The reaction mass was heated to 50-55° C. and maintained for 12 hours. The reaction mass was cooled to 35-40° C. and filtered the reaction mass, washed with isopropyl alcohol (3.4 L), dried at 55-60° C. for 6-8 hours to give the title compound. Yield: 59.27%

### Example-22

### Preparation of ethyl 5-(2-(butoxycarbonyl)-4-phenoxyphenyboxazole-4-carboxylate

Triphenylphosphine (6.03 g) and dichloromethane (30 mL) were charged at 28° C. Triethylamine (4.65 g) and ethyl 2-isocyanoacetate (2 g) were added to the reaction mixture at 28° C. The reaction mixture was cooled to 2° C. Carbon tetrachloride (3.54 g) was added to the reaction mass at 2° C. and maintained for 10-12 hours. The solvent from the reaction mass was completely distilled off and purified by column chromatography to obtain isocyanide compound.

The obtained isocyanide compound and dichloromethane (2 mL) were charged at 24° C. The solution of 2-(butoxycarbonyl)-4-phenoxybenzoic acid (5.56 g) in dichloromethane (2 mL) was added to the above solution at 24° C. and maintained for 8-10 hours. The solvent from the reaction mass was completely distilled off and purified by column chromatography to obtain the title compound.

### Example-23

### Preparation of ethyl 4-hydroxy-1-oxo-7-phenoxy-1,2-dihydro isoquinoline-3-carboxylate

Ethyl 5-(2-(butoxycarbonyl)-4-phenoxyphenyboxazole-4-carboxylate (1 g), methanol (5 mL) and conc. HCl (1 mL) were charged at 28° C. The temperature of the reaction was raised to 65-67° C. and maintained for 3-4 hours. The reaction mass was cooled to 28° C. and stiffed for 5-10 minutes. The obtained solid was filtered and washed with methanol (3 mL), dried at 64° C. for 90 minutes to give the title compound. Yield: 78%

### Example-24

### Preparation of ethyl-4-hydroxy-1-methyl-7-phenoxyisoquinoline-3-carboxylate

Ethyl-1-bromo-4-hydroxy-7-phenoxyisoquinoline-3-carboxylate (1 g), Fe(acac)₃ (0.364 g) were charged at 30° C. N-methyl pyrrolidine (NMP; 2.8 mL), THF (20 mL) were added to the reaction mixture at 30° C. and stirred for 5-10 minutes. The reaction mass was cooled to −60° C. and methyl magnesium chloride (0.867 g) was slowly added to the reaction mass at −60° C. for a period of 15-20 minutes and maintained for 3-4 hours. The reaction mixture was quenched with ethyl acetate (40 mL) and dilute HCl (40 mL) and stirred for 5-10 minutes. Layers were separated and the organic layer was distilled at 45° C. under vacuum. Acetone (5 mL) was added to the resulting residue and stirred for 5-10 minutes. The obtained solid was filtered and washed with acetone (1 mL) to obtain the title compound. Conversion HPLC: 84%

### Example-25

### Purification of ethyl 4-hydroxy-1-methyl-7-phenoxyisoquinoline-3-carboxylate

Ethyl 4-hydroxy-1-methyl-7-phenoxyisoquinoline-3-carboxylate (2 g) and acetone (12 mL) were charged at 28° C. and stirred for 5-10 minutes. The reaction mixture was heated to 50-55° C. and maintained for 30 minutes. The reaction mass was cooled to 25-35° C. and maintained for 60 minutes. The obtained solid was filtered and washed with acetone (2 mL) to obtain the title compound. Purity: 99.39%

### Example-26

### Purification of Roxadustat

Acetone (5 mL) and Roxadustat (0.5 mg) were charged at 28° C. and stirred for 5-10 minutes. The reaction mixture was heated to 50-55° C. and maintained for 30 minutes. The reaction mass was cooled to 25-35° C. and maintained for 30 minutes. The obtained solid was filtered and washed with acetone (1 mL) to obtain the title compound. Purity: 99.59%

### Example-27

### Preparation of 4-Phenoxyphthalic acid

4-Nitro-phthalonitrile (25 g), Toluene (150 mL), DMSO (25 mL), Phenol (16.31 g), potassium carbonate (31.9 g) and DMSO (14.83 g) were charged in RBF at 25-35° C. The reaction mixture was heated to 80-90° C. and maintained for 6-7 hours. DM water (125 mL) was added to the reaction mass and stirred for 10 minutes. Layers were separated. Cool the aqueous layer to 25-35° C. DM water (150 mL) and KOH (40.4 g) were added to the aq. layer. The reaction mixture was heated to 80-90° C. and maintained for 10-15 hours. The reaction mixture was cooled to 25-35° C. Layers were separated. DM water (100 mL) was added to the aq. layer and cooled to 10-20° C. Conc. HCl (−50 mL) was slowly added to the reaction mass to adjust the pH to below 2 and stiffed the reaction mass for 1-2 hours at 25-35° C. Filtered the obtained solid and washed with DM water (100 mL), dried at 50-60° C. for 5-6 hours to give the title compound. Yield: 88.5%

### Example-28

### Preparation of 2-(butoxycarbonyl)-4-phenoxybenzoic acid

4-Phenoxyphthalic acid (25 g), Toluene (50 mL), KOH (0.542 g), acetic acid (5 mL) and acetic anhydride (14.83 g) were charged in RBF at 25-35° C. The reaction mixture was heated to 80-90° C. and maintained for 3-4 hours. The reaction mixture was cooled to 25-35° C. n-Butanol (21.53 g) was added to the reaction mass at 25-35° C. and stirred for 12-14 hours. DM water (125 mL) was added to the reaction mass and stirred for 10 minutes. Layers were separated and the organic layer was washed with DM water (125 mL). n-heptane (500 mL) was added to the organic layer and heated to 50-60° C., maintained the reaction mass at 55° C. for 30 minutes. The reaction mass was cooled to 0-5° C. and maintained for 2-3 hours. Filtered the obtained solid and washed with n-heptane (50 mL), dried at 45-50° C. for 5-6 hours to give the title compound. Yield: 61%

### Example-29

### Preparation of ethyl 4-hydroxy-1-oxo-7-phenoxy-1,2-dihydroisoquinoline-3-carboxylate

2-(butoxycarbonyl)-4-phenoxybenzoic acid (50 g), DMF (200 mL) were charged at 26° C. and stiffed for 10 minutes. Di(1H-imidazol-1-yl)methanone (37.4 g) was added to the reaction mixture at 26° C. and heated up to 36° C., maintained for 6-8 hours. The reaction mass was cooled to 5° C. ethyl 2-cyanoacetate (30.5 g) and 2,3,4,6,7,8,9,10-octahydropyrimido[1,2-a]azepine (36.3 g) in DMF (50 mL) were added to the reaction mass at 6° C. and maintained for 6-8 hours. Toluene (250 mL) and water (60 mL) were charged in to the reaction mass and stiffed for 10 minutes. Layers were separated and extracted the aqueous layer with toluene (100 mL). Combine the organic layer and washed with water (120 mL). The solvent from the organic layer was distilled at 65° C. to obtain residue compound. The obtained residue was dissolved in isopropyl alcohol (600 mL) and stirred for 10 minutes. Phosphoric acid (101 g) was added to the reaction mixture at 26° C. The reaction mixture was heated up to 78° C. and maintained for 8-10 hours. Filtered the reaction mass and washed with isopropyl alcohol (2×40 mL) and water (40 mL), dried at 60° C. for 2-4 hours to give the title compound. Yield: 74%

### Example-30

### Preparation of ethyl 1-chloro-4-hydroxy-7-phenoxyisoquinoline-3-carboxylate

Ethyl 4-hydroxy-1-oxo-7-phenoxy-1,2-dihydroisoquinoline-3-carboxylate (75 g), chlorobenzene (750 mL) and DMF (7.5 mL) were charged at 28° C. and stiffed for 10 minutes. POCl₃(42.4 g) was added to the reaction mixture at 28° C. The reaction mixture was heated up to 99° C. and maintained for 5-6 hours. The reaction mass was distilled at 60° C. under vacuum. Chlorobenzene (150 mL) was added to the reaction mass and distilled again at 60° C. under vacuum. Acetonitrile (375 mL) was slowly added to the reaction mass at 50° C. and allowed the reaction mass temperature to 30° C. Water (150 mL) was added to the reaction mass and stirred for 1-2 hours. Filtered the solid and washed with water (375 mL) and further washed with a mixture of acetonitrile: water (300 mL (1:1)), dried at 65° C. under vacuum for 5-6 hours to give the title compound. Yield: 90%

### Example-31

### Preparation of ethyl 4-hydroxy-1-methyl-7-phenoxyisoquinoline-3-carboxylate

Ethyl 1-chloro-4-hydroxy-7-phenoxyisoquinoline-3-carboxylate (5 g), Fe(acac)₃ (3.08 g), THF (15 mL) and n-methyl pyrrolidine (35 mL) were charged at 28° C. and stiffed for 10 minutes. The reaction mass was cooled to −2° C. and methyl magnesium chloride (4.90 g) in THF (15 mL) was slowly added to the reaction mass at −2° C. for one hour, maintained for 3-4 hours. Ethyl acetate (50 mL) was added to reaction mass at 4° C. and stiffed for 10 minutes, dil. HCl (50 mL) was also added to the reaction mass. Layers were separated and the organic layer was washed with EDTA tetrasodium salt solution (100 mL) and water (50 mL). The organic layer was charged with anhydrous MgSO₄ (3 g) and stiffed for 10 minutes. Filtered the obtained organic layer. Ethyl acetate (30 mL) was added to the aqueous layer and washed with water (20 mL). Combined the organic layer and distilled at 48° C. under vacuum to obtain the crude. Acetone (20 mL) was added to the obtained crude and stirred for 10 minutes. Filtered the obtained solid and washed with acetone (5 mL), dried at 60° C. for 5-6 hours to give the title compound. Yield: 63%

### Example-32

### Preparation of ethyl 4-hydroxy-1-methyl-7-phenoxyisoquinoline-3-carboxylate HCl

Ethyl 1-chloro-4-hydroxy-7-phenoxyisoquinoline-3-carboxylate (20 g), n-methyl pyrrolidine (240 mL) were charged at 28° C. Fe(acac)₃ (12.33 g), THF (80 mL) were charged to the reaction mass at 28° C. and stirred for 15 minutes. The reaction mass was cooled to 7° C. and methyl magnesium chloride (19.58 g) was slowly added to the reaction mass at 7° C. for two hours, maintained for 1-2 hours. The reaction mass temperature was cooled to −2° C. and water (40 mL) was slowly added to the reaction mass. 20% Aq hydrochloric solution (200 mL) was added to the reaction mass at 2° C. The reaction mass temperature was raised to 29° C. and maintained for 2-3 hours. Toluene (200 mL) was added to the reaction mass at 28° C. and stiffed for 10 minutes. Layers were separated and the aqueous layer was washed with toluene (2×100 mL). Organic layer was washed with aqueous hydrochloric solution (100 mL), Again organic layer washed with water (100 mL). The organic layer was distilled at 55° C. under vacuum. toluene (40 mL) was added to the obtained crude and it was heated to 60° C. The reaction mass was cooled to 28° C. Acetone (40 mL) and IPA. HC1 (12.5 mL) were slowly added to reaction mass and maintained for 2 hours. The reaction mass was cooled to 5° C. Filtered the obtained solid and washed with acetone (40 mL), dried at 62° C. for 3-4 hours to give the title compound.

### Example-33

### Preparation of ethyl-4-hydroxy-1-methyl-7-phenoxyisoquinoline-3-carboxylate HCl

Ethyl 4-hydroxy-1-methyl-7-phenoxyisoquinoline-3-carboxylate (20 g), THF (50 mL) and Con HCl (5 mL) were charged at 28° C. and stirred for 10 minutes. The reaction mass is maintained for 2-3 hours. Filtered the obtained solid and dried at 55° C. for 4-5 hours to give the title compound. Yield: 52.5%

### Example-34

### Preparation of ethyl-4-hydroxy-1-methyl-7-phenoxyisoquinoline-3-carboxylate 4-methylbenzenesulfonate

Ethyl 4-hydroxy-1-methyl-7-phenoxyisoquinoline-3-carboxylate (1 g), 1,4-dioxane (10 mL) and PTSA monohydrate (1.175 g) were charged at 28° C. and stiffed for 10 minutes. The reaction mass is maintained for 2-3 hours. Filtered the obtained solid and dried at 50° C. for 4-5 hours to give the title compound. Yield: 90%

### Example-35

### Preparation of ethyl-4-hydroxy-1-methyl-7-phenoxyisoquinoline-3-carboxylate sulfate

Ethyl 4-hydroxy-1-methyl-7-phenoxyisoquinoline-3-carboxylate (1 g), 1,4-dioxane (10 mL) and H₂SO₄ (0.606 g) were charged at 28° C. and stiffed for 10 minutes. The reaction mass is maintained for 1-2 hours. Filtered the obtained solid and dried at 50° C. for 4-5 hours to give the title compound.

### Example-36

### Preparation of Roxadustat

Ethyl 4-hydroxy-1-methyl-7-phenoxyisoquinoline-3-carboxylate (30 g), dimethyl formamide (90 mL), glycine (10.5 g) and DBU (21.19 g) were charged at 28° C. and stirred for 10 minutes. The reaction mass was heated up to 73° C. and maintained for 3-4 hours. The reaction mass was cooled to 28° C. Water (120 mL) was added to the reaction mass and stiffed for 10 minutes. Layers were separated and the aqueous layer was washed with toluene (2×150 mL). Acetonitrile (150 mL) was added to the aqueous layer and stiffed for 10 minutes. The reaction mass was adjusted the pH 3-4 with 10% Aqueous hydrochloric acid (165 mL) and maintained for 2-3 hours. Filtered the obtained solid, washed with water (150 mL) and acetonitrile (60 mL) to give the title compound. Yield: 93%

### Example-37

### Preparation of Roxadustat

Ethyl 4-hydroxy-1-methyl-7-phenoxyisoquinoline-3-carboxylate hydrochloride (5 g), dimethyl formamide (15 mL), glycine (3.13 g) and DBU (10.58 g) were charged at 28° C. and stiffed for 10 minutes. The reaction mass was heated up to 76° C. and maintained for 4-5 hours. The reaction mass was cooled to 28° C. Water (20 mL) and toluene (25 mL) were added to the reaction mass and stirred for 10 minutes. Layers were separated and the aqueous layer was washed with toluene (25 mL). Again layers were separated. Acetonitrile (50 mL) was added to the aqueous layer and stiffed for 10 minutes. Water (5 mL) and conc. HCl (20 mL) were slowly added to the reaction mass and stiffed for 10 minutes. Filtered the obtained solid and washed with water (25 mL) to give wet compound. The obtained wet compound and DMF (10 mL) were charged at 28° C. and stirred for 10 minutes. The reaction mass was heated to 47° C. Acetonitrile (50 mL) was added to the reaction mass at 46° C. The reaction mass was cooled to 30° C. and maintained for 2-3 hours. Filtered the obtained solid and washed with acetonitrile (5 mL) to give the title compound. Yield: 65%

### Example-38

### Preparation of Roxadustat

Ethyl 4-hydroxy-1-methyl-7-phenoxyisoquinoline-3-carboxylate hydrochloride (5 g), dimethyl formamide (15 mL), glycine (3.13 g) and 1,1,3,3-Tetramethyl guanidine (8 g) were charged at 28° C. and stirred for 10 minutes. The reaction mass was heated up to 57° C. and maintained for 5-6 hours. The reaction mass was cooled to 28° C. Water (20 mL) and toluene (25 mL) were added to the reaction mass and stirred for 10 minutes. Layers were separated and the aqueous layer was washed with toluene (5 mL). Again layers were separated. Acetonitrile (25 mL) was added to the aqueous layer and stirred for 10 minutes. Water (25 mL) and conc. HCl (15 mL) were slowly added to the reaction mass and stiffed for 60 minutes. Filtered the obtained solid and washed with water (25 mL) and acetonitrile (10 mL) to give wet compound. The obtained wet compound and DMF (10 mL) were charged at 28° C. and stiffed for 10 minutes. The reaction mass was heated to 48° C. Acetonitrile (50 mL) was added to the reaction mass at 46° C. The reaction mass was cooled to 30° C. and maintained for 2-3 hours. Filtered the obtained solid and washed with acetonitrile (5 mL) to give the title compound. Yield: 70%

We claim:

1) A process for the preparation of Roxadustat (I) or its pharmaceutically acceptable salts, which comprises;
a) converting a compound of formula (II) to compound of formula (III); 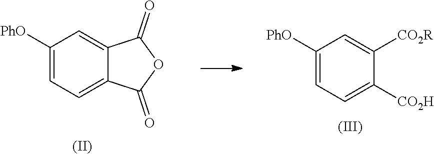
wherein R is C₁-C₆ alkyl;
b) optionally purifying a compound of formula (III);
c) treating a compound of formula (III) with alkyl 2-isocyanoacetate (IV) to form a compound of formula (V); 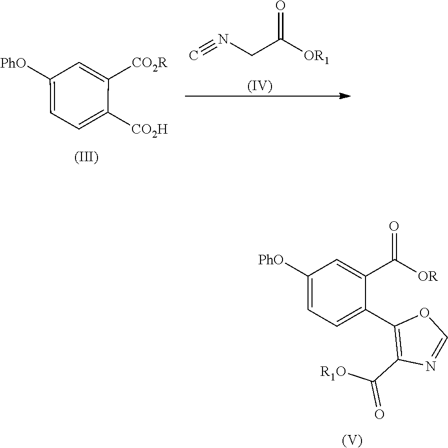
wherein R is C₁-C₆ alkyl and R₁ is H, C₂-C₆ alkyl;
d) converting a compound of formula (V) to form a compound of formula (VI); 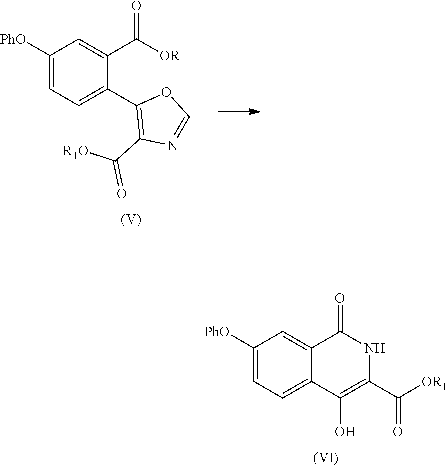
wherein R is C₁-C₆ alkyl and R₁ is H, C₂-C₆ alkyl;
e) halogenation of a compound of formula (VI) to form a compound of formula (VII); 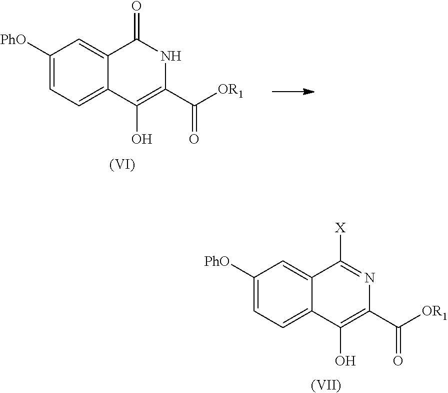
wherein R₁ is H, C₂-C₆ alkyl; X is Cl, Br, I;
f) converting a compound of formula (VII) to a compound of formula (VIII); 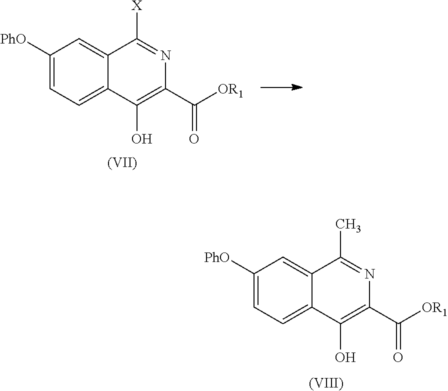
wherein R₁ is H, C₂-C₆ alkyl; X is Cl, Br, I;
g) treating a compound of formula (VIII) with glycine to form Roxadustat (I) or its pharmaceutically acceptable salts.

2) The process according to claim 1, wherein base used in step c) is carried out in presence of a base selected from pyridine, piperidine, pyrimidine, triethylamine, tributylamine, N-methylmorpholine, N,N-diisopropylethylamine, diethylamine, 1,1,3,3-tetramethylguanidine, DBU, DABCO.

3) The process according to claim 1, wherein acid used in step d) is carried out in presence of acid selected from hydrochloric acid, sulphuric acid, hydrobromic acid, orthophosphoric acid, Lewis acids, AlCl₃, FeCl₃, acetic acid, citric acid, oxalic acid, trifluoroacetic acid.

4) A process for the preparation of Roxadustat (I) or its pharmaceutically acceptable salts, which comprises;
a) converting a compound of formula (III) to a compound of formula (IIIa); 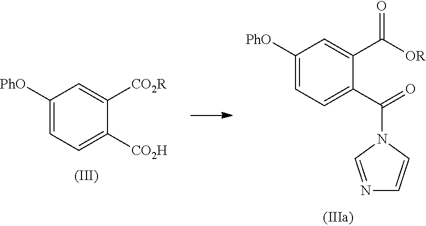
wherein R is C₁-C₆ alkyl;
b) treating a compound of formula (IIIa) with alkyl 2-isocyanoacetate (IV) to form a compound of formula (V); 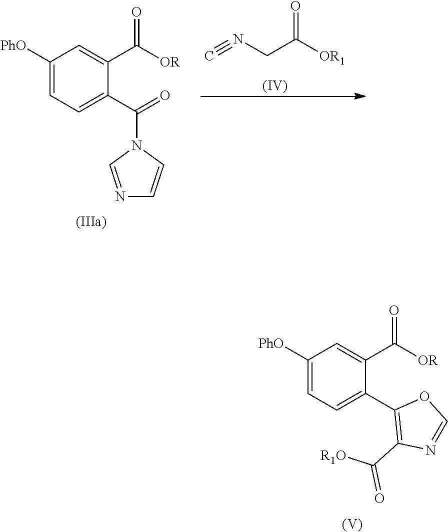
wherein R is C₁-C₆ alkyl and R₁ is H, C₂-C₆ alkyl;
c) converting a compound of formula (V) to Roxadustat (I) or its pharmaceutically acceptable salts.

5) The process according to claim 4, wherein base used in step b) is carried out in presence of a base selected from pyridine, piperidine, pyrimidine, triethylamine, tributylamine, N-methylmorpholine, N,N-diisopropylethylamine, diethylamine, 2,2-bipyridine, 1,1,3,3-tetramethylguanidine, DBU, DABCO.

6) A process for the preparation of Roxadustat (I) or its pharmaceutically acceptable salts, which comprises;
a) treating a compound of formula (VII) with a methylating reagent in presence of catalyst to form a compound of formula (VIII); 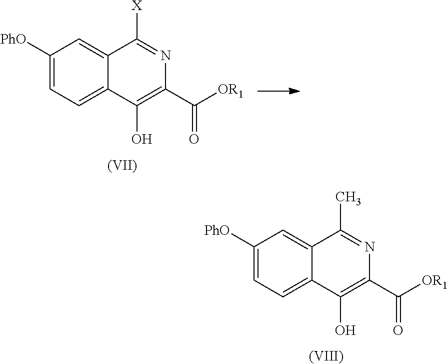
wherein R₁ is H, C₂-C₆ alkyl; X is Cl, Br, I, OTf;
b) treating a compound of formula (VIII) with glycine to form Roxadustat (I) or its pharmaceutically acceptable salts.

7) The process according to claim 6, wherein the methylating reagent used in step a) is selected from trimethyl boroxine, methylmagnesium chloride, methyl magnesium bromide, methyl lithium, trimethyl silyl halides.

8) The process according to claim 6, wherein the catalyst used in step a) is selected from Tris(acetylacetonato)iron(III), triphenylphosphine palladium, CuI, manganese halides, FeCl₃, Nickel halides, Ni(acac)₂, Ni(COD)₂, Cobalt halides.

9) The process according to claim 6, wherein the base used in step a) is carried out in presence of a base selected from pyridine, piperidine, pyrimidine, triethylamine, tributylamine, N-Methyl-2-pyrrolidone (NMP), N-methylmorpholine, DBU, DABCO sodium carbonate, potassium carbonate, sodium bicarbonate, potassium bicarbonate, sodium hydroxide, potassium hydroxide, lithium hydroxide, calcium hydroxide.

10) A process for the preparation of Roxadustat (I) or its pharmaceutically acceptable salts, which comprises;
a) treating a compound of formula (VIII) with acid (HA) to form an acid addition salt of compound of formula (VIIIa); 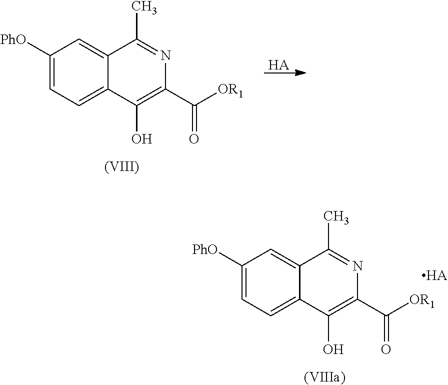
wherein R₁ is H, C₂-C₆ alkyl;
b) converting a compound of formula (VIIIa) to Roxadustat (I) or its pharmaceutically acceptable salts.

11) The process according to claim 10, wherein the acid used in step a) is selected from hydrochloric acid, sulfuric acid, acetic acid, oxalic acid, p-toluene sulfonic acid, oxalic acid, trifluoroacetic acid.

12) A compounds of formula (III), (V), (VI), (VIII), (IX), (X), (XI), (XII), (IIIa), (IIIb), (VIIIa) and (XIIIa). 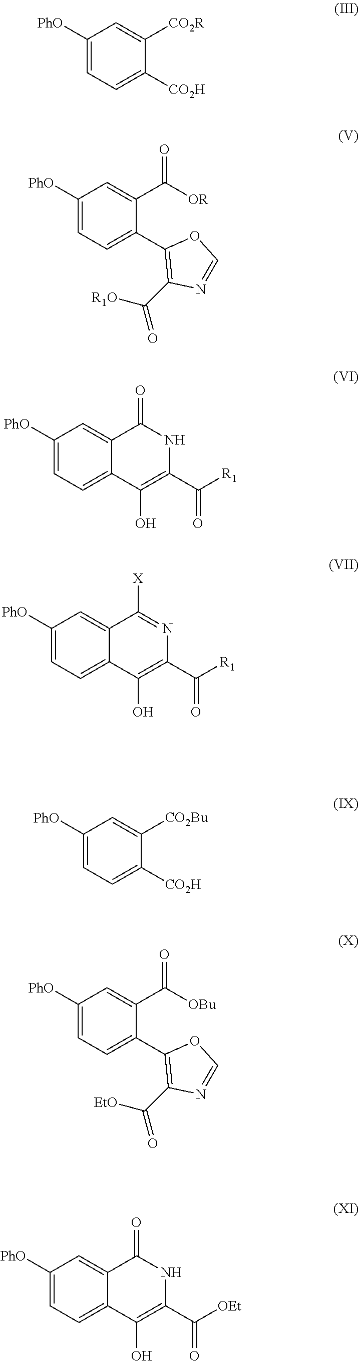 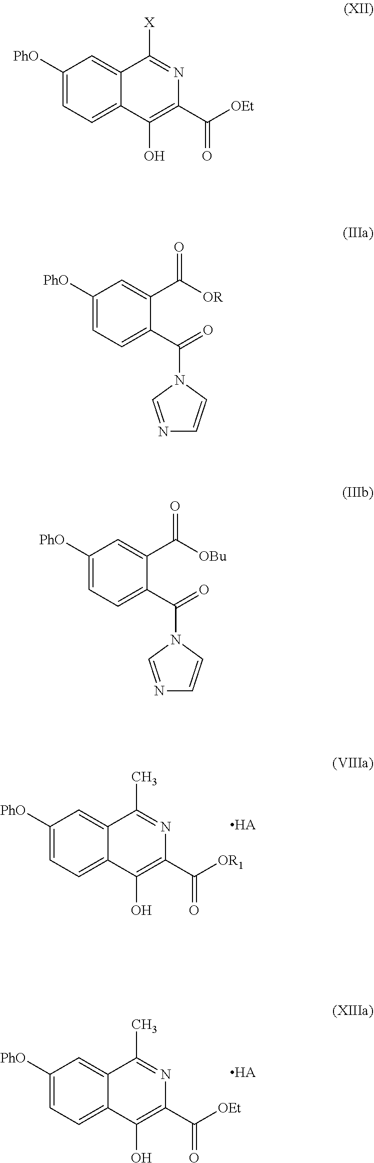
wherein R is C₁-C₆ alkyl, R₁ is H, C₂-C₆ alkyl and X is Cl, Br and I;
